(19) United States
(12) Patent Application Publication
(10) Pub. No.: US 2026/0232763 A1
(43) Pub. Date: Aug. 13, 2026

(54) Methods of Administering Belumosudil in Combination with Transport Protein Substrates

(71) Applicant: Kadmon Corporation, LLC, Morristown, NJ (US)

(72) Inventor: Jasminder Sahi, Cambridge, MA (US)

(73) Assignee: Kadmon Corporation, LLC, Morristown, NJ (US)

(21) Appl. No.: 19/638,495

(22) Filed: Apr. 3, 2026

Related U.S. Application Data
(63) Continuation of application No. PCT/US2024/049962, filed on Oct. 4, 2024.
(60) Provisional application No. 63/588,359, filed on Oct. 6, 2023.

(51) Int. Cl. A61K 38/17; A61K 31/505; A61K 31/517; A61P 37/06

(52) U.S. Cl. CPC A61K 38/177; A61K 31/505; A61K 31/517; A61P 37/06

(57) ABSTRACT

The present disclosure relates to the co-administration of belumosudil and transport protein substrates, such as substrates of organic anion transporting polypeptide 1B1 (OATP1B1), breast cancer resistance protein (BCRP), and P-glycoprotein (P-gp), for use in the treatment of subjects with chronic graft-versus-host disease (cGVHD).

### CROSS-REFERENCE TO RELATED APPLICATIONS

This application claims priority to U.S. Application No. 63/588,359 filed Oct. 6, 2023, which is incorporated herein by reference in its entirety.

### TECHNICAL FIELD

The present disclosure relates to methods of treating chronic graft-versus-host disease (cGVHD) by co-administration of a substrate of organic anion transporting polypeptide 1B1 (OATP1B1), breast cancer resistance protein (BCRP), and/or P-glycoprotein (P-gp), and 2-{3-[4-(1H-indazol-5-ylamino)-2-quinazolinyl]phenoxy}-N-(propan-2-yl) acetamide or pharmaceutically acceptable salts thereof (Compound), wherein the dose of the substrate is reduced as compared to its clinically-recommended dose when the substrate is administered without the belumosudil.

### BACKGROUND

Chronic graft-versus-host disease (cGVHD) is an immune-mediated inflammatory and fibrotic disorder. It is a potential, serious complication following solid organ transplant and allogeneic hematopoietic cell transplant (alloHCT). cGVHD affects up to 70% of all alloHCT recipients, with an incidence of 20%-50% in children. It is the leading cause of non-relapse mortality beyond 2 years after alloHCT. The estimated prevalence of cGVHD is 14,000 patients in the United States (as of 2016). (Bachier C R et al: Epidemiology and real-world treatment of chronic graft-versus-host disease post allogeneic hematopoietic cell transplantation: A US claims analysis. Presented at ASH 2019, Orlando, FL, December 7-10, 2019) (“Bachier et al.”).

Patients with cGVHD have substantial impairment in quality of life (QOL) as assessed by the Lee Symptom Scale (LSS), which measures the effect of cGVHD on patients' functioning and well-being. It is reported that only one third of patients who have cGVHD and start systemic treatment will be alive, in remission and off immunosuppressive therapy by 5 years. (Lee S J et al: Success of immunosuppressive treatments in patients with chronic graft-versus-host disease. Biol Blood Marrow Transpl 24:555-562, 2018) (“Lee et al.”).

The pathophysiology of cGVHD can be separated into three phases: early inflammation because of tissue injury, a dysregulated adaptive immune system, and chronic inflammation and aberrant tissue repair with fibrosis. Management of cGVHD continues to evolve with the advent of targeted therapies.

Along with medication(s) used to treat cGVHD, patients with cGVHD can be administered additional medication to treat side effects of the cGVHD medication and/or to treat non-cGVHD related diseases or disorders. Such additional medication can affect, and be affected by, the medication(s) used to treat the cGVHD. Drug-drug interactions can depend upon the patient's individual metabolic profile, the drugs' mechanism of action, the drugs' biological target, and the patient's nutritional intake. Drug metabolism further complicates these interactions, as a drug metabolite may be the active therapeutic agent of a certain medication.

Drug-drug interactions can cause an increase or a reduction in the patient's exposure to the drugs involved, including to toxic or non-efficacious levels, and are challenging to predict and to resolve. Dose adjustment of the medications may be needed to mediate these interactions, which can lead to difficult trial-and-error periods for cGVHD patients. In some instances, halting treatment with a drug may result.

There remains an opportunity to investigate exposure and dosing of co-administered medications.

### SUMMARY

The present disclosure relates to treating chronic graft-versus-host-disease (cGVHD) in a subject in need thereof, comprising co-administering at least one substrate of organic anion transporting polypeptide 1B1 (OATP1B1), breast cancer resistance protein (BCRP), or P-glycoprotein (P-gp) and 2-{3-[4-(1H-indazol-5-ylamino)-2-quinazolinyl]phenoxy}-N-(propan-2-yl) acetamide, or a pharmaceutically acceptable salt thereof (“Compound”) wherein the dose of the at least one substrate of OATP1B1, BCRP, or P-gp is reduced as compared to the clinically-recommended dose when the substrate is administered without the Compound.

The present disclosure also provides a method for treating a subject with cGVHD comprising the steps of (a) determining whether the subject is in need of co-administration of at least one substrate of OATP1B1, BCRP, or P-gp; (b) determining the clinically-recommended dose of the at least one substrate of OATP1B1, BCRP, or P-gp for the subject when the substrate is administered without co-administration of 2-{3-[4-(1H-indazol-5-ylamino)-2-quinazolinyl]phenoxy}-N-(propan-2-yl) acetamide or a pharmaceutically acceptable salt thereof (“Compound”); (c) determining a lower dose of the at least one substrate of OATP1B1, BCRP, or P-gp to be administered to the subject to mediate the increase in exposure of the substrate due to the administration of the Compound; and (d) co-administering the at least one substrate of OATP1B1, BCRP, or P-gp and the Compound.

The present embodiments can be understood more fully by reference to the detailed description and examples, which are intended to exemplify non-limiting embodiments.

### DETAILED DESCRIPTION

### Overview

cGVHD is characterized by an overproduction of proinflammatory cytokines IL-21 and IL-17, as well as overactivation of T follicular helper cells and B cells, which in turn leads to overproduction of antibodies. By controlling ROCK2 activity, belumosudil mediates signaling in immune cellular function and fibrotic pathways, thereby alleviating the effects caused by this debilitating disease, such as inflammation of multiple tissues and fibrotic changes that may involve several organs including the lungs, hepatobiliary system, musculoskeletal system, gastrointestinal (GI) tract, and skin.

In vitro assessments have suggested that metabolism of belumosudil is primarily dependent on cytochrome P450 CYP3A4 activity and that the solubility of belumosudil is pH dependent. Preclinical investigations in mouse, rat, rabbit, and dog have indicated that belumosudil undergoes hepatic metabolism to form 2 main metabolites called KD025m1 (a ROCK2 active minor metabolite) and KD025m2 (a ROCK2 inactive major metabolite). These main metabolites were subsequently quantified in clinical trials across the belumosudil development program; exposure of the major, inactive metabolite KD025m2 was 15% to 20% of the parent, while the minor, active metabolite KD025m1 had exposure value <5% of the parent.

It was determined that belumosudil may affect exposure of co-administered drugs, including drugs that are substrates of transport proteins. Transport proteins can move material across a biological membrane, including to facilitate pumping drugs into or out of a cell against their concentration gradient. Transport proteins include the ATP-binding cassette (ABC) transporter family, of which breast cancer resistance protein (BCRP) and P-glycoprotein (P-gp) are members. The solute carrier proteins (SLC) are uptake transporters, such as organic anion transporting polypeptides (OATPs) that include organic anion transporting polypeptide 1B1 (OATP1B1). OATP1B1 is highly expressed in human liver and can extract its substrates from circulating blood into, for example, hepatocytes. Once in a cell, a transport protein substrate can then be subject to further transport or be metabolized, and metabolites may serve as transport protein substrates as well. Thus, drugs that are substrates or inhibitors of transport proteins can affect the drug-drug interactions in patients, including patients that take more than one medication to manage, for example, chronic disease.

As such, a three-part, sequential, non-randomized, open-label study was conducted in healthy volunteers, to evaluate the effect of oral Belumosudil on inhibition of the phase 2 drug metabolizing enzyme UGT1A1 and the transporters P-gp, BCRP and OATP1B1, which is described in Example 3. This study involved three parts, with Part 1 conducted to assess the effect of multiple doses of belumosudil on the single-dose PK of raltegravir (a UGT1A1-sensitive substrate), Part 2 conducted to assess the effect of multiple doses of belumosudil on the single-dose PK of dabigatran etexilate (a P-gp-sensitive substrate), and Part 3 conducted to assess the effect of multiple doses of belumosudil on the single-dose PK of rosuvastatin (a BCRP/OATP1B1-sensitive substrate).

Administration of belumosudil with each of the co-administered drugs was safe, and no notable adverse events were reported.

Co-administration of belumosudil with a drug that is a substrate of these transport proteins can increase exposure of the substrate. The dosage of the substrate can therefore be decreased when co-administered with belumosudil. In an embodiment, the dosage of a transport protein substrate is decreased when co-administered with belumosudil. For example, in one embodiment, the dosage of rosuvastatin is decreased from a clinically-recommended dosage when co-administered with a clinically-recommended dosage of belumosudil. In an embodiment, the dosage of rosuvastatin is decreased to about 5 mg daily when co-administered with about 200 mg of belumosudil.

### Definitions

“About” as used herein includes the exact amount modified by the term, about, as well as an amount that would be expected to be within experimental error, such as for example, within 15%, 10%, or 5%. For example, “about 200 mg” means “200 mg” and also a range of mgs that is within experimental error, e.g., plus or minus 15%, 10%, or 5% of 200 mg. As used herein, the term “about” may be used to modify a range and also, a particular value.

“Administering” or “administered to” as used herein (for example, with reference to administration of APIs, including “co-administration” of one or more APIs, such as Compound, belumosudil, and/or a substrate of OATP1B1, BCRP, or P-gp to a subject), refers to the act of prescribing medicine(s) containing one or more of the APIs for the subject to take during treatment, the act of prescribing a protocol of medicines to be taken by a subject, the act of dispensing the medicine(s) to the subject, and/or the act of physically receiving or ingesting the medicine(s). Thus, the APIs (e.g., Compound, belumosudil, and/or a substrate of OATP1B1, BCRP, or P-gp), can be “administered” by a physician or other medical professional who writes prescriptions for any one of such medicine(s); and/or by a pharmacist who fills said prescriptions and/or dispenses one or more of the medicine(s) to the subject; and/or by the patient or subject who ingests the medicine(s) and/or his or her partner or caretaker who delivers the medicine(s) to the subject.

“API” means “active pharmaceutical ingredient.”

“Allogeneic hematopoietic stem cell transplantation (allo-HSCT)” also called bone marrow transplantation or stem cell transplantation, or “allogeneic hematopoietic cell transplantation (allo-HCT)” refers to a procedure where hematopoietic cells from a donor are grafted into a recipient who is not an identical twin. The source of hematopoietic stem cells for allogeneic transplantation may be peripheral blood stem cells (PBSC) or bone marrow (BM). In some circumstances umbilical cord blood may be used. The donor and recipient may be matched at the human leukocyte antigen (HLA) genes, such as siblings. The donor and recipient may be a parent and a child who are only half-matched (haploidentical).

Belumosudil is an oral selective rho-associated coiled-coil-containing protein kinase-2 (ROCK2) inhibitor. ROCK2 inhibition acts on the dysregulated adaptive immune system and the fibrosis that occurs as a result of aberrant tissue repair. Belumosudil inhibits ROCK2 and ROCK1 with IC50 values of approximately 100 nM and 3 μM, respectively. Belumosudil down-regulated proinflammatory responses via regulation of STAT3/STAT5 phosphorylation and shifting Th17/Treg balance in ex-vivo or in vitro-human T cell assays.

The compound belumosudil has the chemical name: 2-{3-[4-(1H-indazol-5-ylamino)-2-quinazolinyl]phenoxy}-N-(propan-2-yl) acetamide. The compound belumosudil is also known as KD025. The mesylate salt of belumosudil is marketed as REZUROCK™ in the United States for the treatment of patients with chronic GVHD after failure of at least two prior lines of systemic therapy. The active pharmaceutical ingredient of REZUROCK™ is belumosudil mesylate salt with the molecular formula C₂₇H₂₈N₆O₅S, a molecular weight of 548.62 g/mol, and having the chemical name 2-{3-[4-(1H-indazol-5-ylamino)-2-quinazolinyl]phenoxy}-N-(propan-2-yl) acetamide methanesulfonate (1:1).

The chemical structure of belumosudil mesylate is as follows:

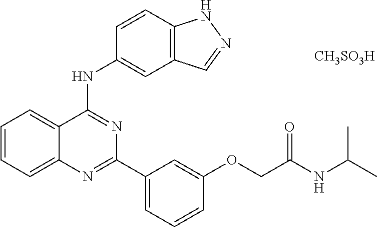 [SMILES: CC(C)NC(=O)COc1cccc(-c2nc(Nc3ccc4[nH]ncc4c3)c3ccccc3n2)c1 ; "CH3SO3H"]

Belumosudil and processes for making the compound are described in the following US patents: U.S. Pat. Nos. 8,357,693, 9,815,820, 10,183,931, and 10,696,660.

When the term “Belumosudil” is used herein, it should be understood that, unless the context clearly indicates otherwise, the term may cover the compound belumosudil in any form as well as pharmaceutically acceptable salts thereof. The term “Belumosudil” refers both to the compound belumosudil (for example, in the free base form, amorphous form, or crystalline form), to pharmaceutically acceptable salts of belumosudil, for example, the mesylate salt form as used in REZUROCK,™ and to any form of belumosudil that may be used in a formulation or pharmaceutical composition for administering the compound to a patient.

“BCRP” refers to breast cancer resistance protein, a membrane transport protein that is a member of the ATP-binding cassette (ABC) transporter family and is encoded by the ABCG2 gene. BCRP is also known as ATP-binding cassette super-family G member 2 (ABCG2) and CDw338. Substrates of BCRP include 2-amino-1-methyl-6-phenylimidazo[4,5-b]pyridine (PhIP), estradiol-17-beta-glucuronide, estrone-3-sulfate, methotrexate, rosuvastatin, prazosin, and sulfasalazine.

“Clinical endpoint” or “study endpoint” refers to an event or outcome in a clinical trial that can be measured objectively to determine outcomes and potential beneficial effects of the drug or administration protocol as designed in the clinical trial.

“Clinically recommended amount” or “clinically recommended dosage” refers to the amount or dosage of API that has been recommended and/or approved for administration to a subject by those skilled in the field of medicinal chemistry to treat the disease state in question following clinical trials, for example, as set forth in publications, clinical trial results, and on the approved drug label. In one embodiment, the clinically recommended dosage is expressed for an API as a free base form. In one embodiment, the clinically recommended dosage is expressed for an API as a free acid, or protonated, form. In one embodiment, a clinically recommended dosage for belumosudil is about 200 mg once daily. In one embodiment, a clinically recommended dosage for belumosudil is about 200 mg twice daily. In one embodiment, a clinically recommended dosage for raltegravir is about 400 mg once daily. In one embodiment, a clinically recommended dosage for raltegravir is about 400 mg twice daily. In one embodiment, a clinically recommended dosage for dabigatran, is about 150 mg twice daily. In one embodiment, a clinically recommended dosage for dabigatran, is about 75 mg twice daily. In one embodiment, a clinically recommended dosage for dabigatran, is about 75 mg once daily. In one embodiment, a clinically recommended dosage for rosuvastatin, is between about 5 to 40 mg once daily. In one embodiment, a clinically recommended dosage for rosuvastatin, is between about 5 to 20 mg once daily.

“Co-administration,” “in combination with,” and/or “co-administered,” as used herein with reference to administration of belumosudil with at least one transport protein substrates means that during the course of the patient's treatment with belumosudil (also referred to herein as the “perpetrator compound”), the patient is also receiving one or more dosages of one or more transport protein substrate (also referred to herein as “victim compounds”). The transport protein substrate needs not be administered concomitantly with, or on the same day, as the belumosudil to be considered as being “co-administered” under this definition. The transport protein substrate may be administered at the same time as the belumosudil, for a number of days prior to the administration of belumosudil, and/or during the course of treatment to be “co-administered.” A transport protein substrate will not be considered “co-administered” or administered “in combination with” the belumosudil if there is a sufficient washout period between administrations to account for a minimum of 5 half-lives of the active moieties dosed.

In an embodiment, the co-administered transport protein substrate comprises a substrate of a member of the ATP-binding cassette (ABC) transporter family or a member of the solute carrier family (SLC). In an embodiment, the co-administered transport protein substrate comprises a substrate of a member of the ATP-binding cassette (ABC) transporter family.

“Compound” as used in the Claims and Embodiments herein, and when apparent from context of usage, is synonymous with the above all-inclusive definition of belumosudil.

“Concurrent administration,” “concurrently” and/or “concurrently taking” with reference to administration of APIs herein (e.g., as applied to a transport protein substrate “concurrently” administered with belumosudil), is synonymous with “co-administered,” as defined above.

“CYP3A” refers to the CYP3A family of P450 isoenzymes including CYP3A4.

When the term “dabigatran” is used herein, it should be understood that, unless the context clearly indicates otherwise, the term may cover the compound dabigatran etexilate in any form as well as pharmaceutically acceptable salts thereof. The term “dabigatran” refers to the compound dabigatran, to pharmaceutically acceptable salts of dabigatran, for example, the mesylate salt form as used in PRADAXA,® and to any form of dabigatran that may be used in a formulation or pharmaceutical composition for administering the compound to a patient.

“Exposure effect” or “exposure” as used herein refers to the impact of belumosudil on the pharmacokinetics (PK) of a transport protein substrate, for example, exposure levels (overall and peak) for substrates and their metabolites. The exposure may also refer to the impact of a transport protein substrate, inhibitor, and/or co-substrate on the PK of belumosudil and its metabolites and rates of elimination of belumosudil and its major metabolites, KD025m1 and KD025m2, when apparent from context of usage. For example, co-administration of belumosudil can increase the exposure of a substrate of a transport protein, as determined by an increase in the Cmax and/or AUC of the substrate. Excessive exposure can lead to toxic levels of belumosudil or of a substrate of a transport protein, or of a metabolite thereof. Reference is further made to Example 3 hereof as exemplification of the term “exposure effect.”

A high-fat, high-calorie meal, as used herein, means a meal containing about 800 to 1,000 calories with approximately 50% of total caloric content of the meal from fat food. For example, in one embodiment, a high-fat breakfast may consist of hash browns, bacon, fried egg, white bread, and 240 mL of full fat milk.

Lee Symptom Scale (LSS) summary score measures the effect on patients' functioning and well-being. The Lee Symptom Scale is a 30-item scale developed to measure the symptoms of cGVHD and is described in Lee S J, et al., Development and validation of a scale to measure symptoms of chronic graft-versus host disease. Biol Blood Marrow Transplant 2002; 8:444-452.

“Mediate” as used herein refers to regulating or compensating for a change in pharmacological exposure of an API based on the effect of a co-administered medication. For example, co-administration of belumosudil can increase the exposure of a substrate of a transport protein. In an embodiment, mediating the increase in exposure of a transport protein substrate when co-administered with belumosudil comprises lowering the administered dose of the substrate.

“OATP1B1” refers to organic anion transporting polypeptide 1B1, a membrane transport protein that is a member of the solute carrier family (SLC) and is encoded by the SLCO1B1 gene. Substrates of OATP1B1 include estradiol-17β-glucuronide, pitavastatin, pravastatin, and rosuvastatin.

“Or” is used in the inclusive sense (equivalent to “and/or”) unless the context requires otherwise.

“Patient” or “subject” as used herein includes an animal or a human; in one embodiment, the term “patient” refers to a human subject.

“P-glycoprotein” and “P-gp” refer to permeability glycoprotein 1, a membrane transport protein that is a member of the ATP-binding cassette (ABC) transporter family and is encoded by the ABCB1 gene. P-gp is also known as ATP-binding cassette sub-family B member 1 (ABCB1), multidrug resistance protein 1 (MDR1), and CD243. Substrates of P-gp include digoxin, fexofenadine, loperamide, N-methylquinidine (NMQ), quinidine, talinolol, vinblastine, and dabigatran etexilate.

“Pharmaceutically acceptable salt” refers to salts that are, within the scope of sound medical judgment, suitable for administration to a subject without undue toxicity, irritation, allergic response or other undesired effect, commensurate with a reasonable benefit/risk ratio. Pharmaceutically acceptable salts of the compounds described herein include those derived from suitable inorganic and organic acids and bases. Such salts include salts formed with inorganic acids, with organic acids such as methanesulfonic acid, and salts formed when an acidic proton present in the parent compound either is replaced by a metal ion, e.g., an alkali metal ion or an alkaline earth ion, including those having potassium or calcium cations.

“Protocol” as used herein refers to the methods or plan that is used to administer one or more APIs to a subject in need of treatment. The term “protocol” is intended to encompass the overall, detailed plan of care for a patient, as well as individual or partial steps that are part of the overall plan. For example, a protocol may include the dosages used for each API the patient will be (or is) receiving, the combination of APIs the patient receives, the timing and method of administration of each API (for example, considering DDIs, food effects, and impact different formulations or modes of delivery may have on absorption and bioavailability), and management of side effects, as well as the overall plan encompassing the dosages, combinations, timing and methods of administration, and side effects, considered together.

When the term “raltegravir” is used herein, it should be understood that, unless the context clearly indicates otherwise, the term may cover the compound raltegravir in any form as well as pharmaceutically acceptable salts thereof. The term “raltegravir” refers to the compound raltegravir, to pharmaceutically acceptable salts of raltegravir, for example, the potassium salt form as used in ISENTRESS, and to any form of raltegravir that may be used in a formulation or pharmaceutical composition for administering the compound to a patient.

When the term “rosuvastatin” is used herein, it should be understood that, unless the context clearly indicates otherwise, the term may cover the compound rosuvastatin in any form as well as pharmaceutically acceptable salts thereof. The term “rosuvastatin” refers to the compound rosuvastatin, to pharmaceutically acceptable salts of rosuvastatin, for example, the calcium salt form as used in CRESTOR, and to any form of rosuvastatin that may be used in a formulation or pharmaceutical composition for administering the compound to a patient.

“Standard treatment conditions” refers to treatment and/or dosage regimes for administration of belumosudil to a patient for treatment of cGVHD, and also to treatment and/or dosage regimes for administration of a transport protein substrate to a patient, pursuant to the above definition of “co-administered.”

“Substrate” or “Transport Protein Substrate” refers to a substrate of a transport protein that is selectively transported by the protein. Transport proteins cause movement of a material, such as a small molecule API, across a biological membrane. As transport proteins move materials across membranes, they can change the concentrations of materials on either side of the membrane, including against their concentration gradient, which impacts the pharmacokinetics and pharmacodynamics of orally delivered medicines. Substrates of transport proteins are compounds that are selectively transported by the protein. Examples of transport proteins include organic anion transporting polypeptide 1B1 (OATP1B1), breast cancer resistance protein (BCRP) and P-glycoprotein (P-gp). Transport proteins can share substrates, for example, rosuvastatin is a substrate of OATP1B1 and also of BCRP.

A substrate of a transport protein may be a drug or a drug metabolite. In an embodiment, a drug co-administered with belumosudil is transported by OATP1B1, BCRP, or P-gp. In an embodiment, a drug co-administered with belumosudil is transported by OATP1B1 or BCRP. In an embodiment, a co drug co-administered with belumosudil is transported by P-gp. In an embodiment, a drug co-administered with belumosudil is transported by OATP1B1 and BCRP. In an embodiment, rosuvastatin co-administered with belumosudil is transported by OATP1B1 and BCRP.

In an embodiment, a transport protein substrate is a substrate of at least one of OATP1B1, BCRP, or P-gp. In an embodiment, a transport protein substrate is a substrate of at least one of OATP1B1, BCRP, and P-gp. In an embodiment, a transport protein substrate is a substrate of one or more of OATP1B1, BCRP, and P-gp. In an embodiment, a transport protein substrate is a substrate of P-gp. In an embodiment, a transport protein substrate is a substrate of OATP1B1 and BCRP. In an embodiment, a transport protein substrate is a substrate of OATP1B1, a substrate of BCRP, or a substrate of P-gp.

In some embodiments, the transport protein substrate is selected from estradiol-17β-glucuronide, pitavastatin, pravastatin, rosuvastatin, 2-amino-1-methyl-6-phenylimidazo[4,5-b]pyridine (PhIP), estradiol-17-beta-glucuronide, estrone-3-sulfate, methotrexate, prazosin, sulfasalazine, digoxin, fexofenadine, loperamide, N-methylquinidine (NMQ), quinidine, talinolol, vinblastine, and dabigatran etexilate. In an embodiment, the transport protein substrate is rosuvastatin or dabigatran etexilate.

A “therapeutically effective amount” of an API means an amount which, when administered to a human for treating a disease (for example, cGVHD), is sufficient to effect treatment for the disease state being treated. As applied to cGVHD in a human, “treating” or “treatment” includes (1) reducing the risk of developing cGVHD and/or inhibiting cGVHD, i.e., arresting or reducing the development of cGVHD or its clinical symptoms; and (2) relieving cGVHD, i.e., causing regression, reversal, or amelioration of the cGVHD or reducing the number, frequency, duration or severity of its clinical symptoms.

The therapeutically effective amount of an API may vary depending upon the health and physical condition of the subject to be treated, the extent of disease progression, the assessment of the medical situation, and other relevant factors. It is expected that the therapeutically effective amount may fall within a range that can be determined through trial and through reference to clinical trial data and results, for example, as described in Example 3 hereof and in scientific literature.

### EXEMPLARY EMBODIMENTS

In some embodiments, there is provided 2-{3-[4-(1H-indazol-5-ylamino)-2-quinazolinyl]phenoxy}-N-(propan-2-yl) acetamide or a pharmaceutically acceptable salt thereof (Compound) for use in the treatment of chronic graft-versus-host-disease (cGVHD) in a subject, wherein at least one substrate of OATP1B1, BCRP, or P-gp is co-administered to the subject, and wherein the dose of the at least one substrate of OATP1B1, BCRP, or P-gp is reduced as compared to the clinically-recommended dose when the substrate is administered without the Compound.

In some embodiments, there is provided 2-{3-[4-(1H-indazol-5-ylamino)-2-quinazolinyl]phenoxy}-N-(propan-2-yl) acetamide or a pharmaceutically acceptable salt thereof (Compound) for use in the treatment of chronic graft-versus-host-disease (cGVHD) in a subject, wherein at least one substrate of UGT1A1 is co-administered to the subject, and wherein the dose of the at least one substrate of UGT1A1 is reduced as compared to the clinically-recommended dose when the substrate is administered without the Compound. In an embodiment, the medication co-administered with the Compound comprises raltegravir.

In another embodiment, a method of treating chronic graft-versus-host-disease (cGVHD) in a subject in need thereof is provided, the method comprising co-administering at least one substrate of OATP1B1, BCRP, or P-gp and 2-{3-[4-(1H-indazol-5-ylamino)-2-quinazolinyl]phenoxy}-N-(propan-2-yl) acetamide or a pharmaceutically acceptable salt thereof (Compound), wherein the dose of the at least one substrate of OATP1B1, BCRP, or P-gp is reduced as compared to the clinically-recommended dose when the substrate is administered without the Compound.

In another embodiment, a method of treating chronic graft-versus-host-disease (cGVHD) in a subject in need thereof is provided, the method comprising co-administering at least one substrate of the enzyme UGT1A1 and 2-{3-[4-(1H-indazol-5-ylamino)-2-quinazolinyl]phenoxy}-N-(propan-2-yl) acetamide or a pharmaceutically acceptable salt thereof (Compound), wherein the dose of the at least one substrate of UGT1A1 is reduced as compared to the clinically-recommended dose when the substrate is administered without the Compound. In an embodiment, the substrate of UGT1A1 is raltegravir.

In another embodiment, a method of treating chronic graft-versus-host-disease (cGVHD) in a subject in need thereof is provided, the method comprising co-administering a medication that comprises a substrate of OATP1B1, a substrate of BCRP, or a substrate of P-gp, with 2-{3-[4-(1H-indazol-5-ylamino)-2-quinazolinyl]phenoxy}-N-(propan-2-yl) acetamide or a pharmaceutically acceptable salt thereof (Compound), wherein the dose of the medication is reduced as compared to the clinically-recommended dose when the medication is administered without the Compound.

For example, the medication co-administered with the Compound is selected from estradiol-17β-glucuronide, pitavastatin, pravastatin, rosuvastatin, 2-amino-1-methyl-6-phenylimidazo[4,5-b]pyridine (PhIP), estradiol-17-beta-glucuronide, estrone-3-sulfate, methotrexate, prazosin, sulfasalazine, digoxin, fexofenadine, loperamide, N-methylquinidine (NMQ), quinidine, talinolol, vinblastine, and dabigatran etexilate. In an embodiment, the medication co-administered with the Compound comprises rosuvastatin or dabigatran etexilate.

In an embodiment, the Compound comprises a pharmaceutically acceptable salt of belumosudil. In an embodiment, the Compound comprises belumosudil mesylate salt.

In an embodiment, the dose of the Compound is about 200 mg.

In some embodiments, the Compound is administered once daily.

In some embodiments, the Compound is administered twice daily.

In some embodiments, the Compound is administered orally.

In an embodiment, a method of treating chronic graft-versus-host-disease (cGVHD) in a subject in need thereof is provided, the method comprising co-administering a substrate of OATP1B1 selected from estradiol-17β-glucuronide, pitavastatin, pravastatin, and rosuvastatin; with 2-{3-[4-(1H-indazol-5-ylamino)-2-quinazolinyl]phenoxy}-N-(propan-2-yl) acetamide or a pharmaceutically acceptable salt thereof (Compound), wherein the dose of the OATP1B1 substrate is reduced as compared to the clinically-recommended dose when the substrate is administered without the Compound.

In an embodiment, a method of treating chronic graft-versus-host-disease (cGVHD) in a subject in need thereof is provided, the method comprising co-administering rosuvastatin and 2-{3-[4-(1H-indazol-5-ylamino)-2-quinazolinyl]phenoxy}-N-(propan-2-yl) acetamide or a pharmaceutically acceptable salt thereof (Compound), wherein the dose of rosuvastatin is reduced as compared to the clinically-recommended dose when the substrate is administered without the Compound.

In an embodiment, a method of treating chronic graft-versus-host-disease (cGVHD) in a subject in need thereof is provided, the method comprising co-administering a substrate of BCRP selected from 2-amino-1-methyl-6-phenylimidazo[4,5-b]pyridine (PhIP), estradiol-17-beta-glucuronide, estrone-3-sulfate, methotrexate, rosuvastatin, prazosin, and sulfasalazine; with 2-{3-[4-(1H-indazol-5-ylamino)-2-quinazolinyl]phenoxy}-N-(propan-2-yl) acetamide or a pharmaceutically acceptable salt thereof (Compound), wherein the dose of the BCRP substrate is reduced as compared to the clinically-recommended dose when the substrate is administered without the Compound.

In an embodiment, a method of treating chronic graft-versus-host-disease (cGVHD) in a subject in need thereof is provided, the method comprising co-administering a substrate of P-gp selected from digoxin, fexofenadine, loperamide, N-methylquinidine (NMQ), quinidine, talinolol, vinblastine, or dabigatran etexilate; with 2-{3-[4-(1H-indazol-5-ylamino)-2-quinazolinyl]phenoxy}-N-(propan-2-yl) acetamide or a pharmaceutically acceptable salt thereof (Compound), wherein the dose of the P-gp substrate is reduced as compared to the clinically-recommended dose when the substrate is administered without the Compound.

In another embodiment, a method of treating chronic graft-versus-host-disease (cGVHD) in a subject in need thereof is provided, the method comprising co-administering at least one substrate of OATP1B1, BCRP, or P-gp and about 200 mg of belumosudil mesylate salt, wherein the dose of the at least one substrate of OATP1B1, BCRP, or P-gp is reduced as compared to the clinically-recommended dose when the substrate is administered without belumosudil mesylate salt.

In some embodiments, the dose of the at least one substrate of OATP1B1, BCRP, or P-gp is reduced by about half as compared to the clinically-recommended dose when the substrate is administered without the Compound. In some embodiments, the dose of the at least one substrate of OATP1B1, BCRP, or P-gp is reduced by about 50% as compared to the clinically-recommended dose when the substrate is administered without the Compound. In some embodiments, the dose of the at least one substrate of OATP1B1, BCRP, or P-gp is reduced by about 10%, 20%, 30%, 40%, 50%, 60%, 70%, 80%, or 90%, as compared to the clinically-recommended dose when the substrate is administered without the Compound.

In some embodiments, the dose of the at least one substrate of OATP1B1, BCRP, or P-gp is reduced by at least about 10%, 20%, 30%, 40%, 50%, 60%, 70%, 80%, or 90%, as compared to the clinically-recommended dose when the substrate is administered without the Compound. In some embodiments, the dose of the at least one substrate of OATP1B1, BCRP, or P-gp is reduced by not more than about 10%, 20%, 30%, 40%, 50%, 60%, 70%, 80%, or 90%, as compared to the clinically-recommended dose when the substrate is administered without the Compound.

In another embodiment, a substrate of OATP1B1 is co-administered to the subject with the Compound.

In another embodiment, the substrate of OATP1B1 is at least one of estradiol-17β-glucuronide, pitavastatin, pravastatin, or rosuvastatin. In another embodiment, the substrate of OATP1B1 is at least one of estradiol-17β-glucuronide, pitavastatin, pravastatin, rosuvastatin, atorvastatin, lovastatin, or simvastatin. In another embodiment, the substrate of OATP1B1 is a statin. In another embodiment, the substrate of OATP1B1 is at least one of atorvastatin, lovastatin, pitavastatin, rosuvastatin, or simvastatin. In an embodiment, the substrate of OATP1B1 is atorvastatin. In an embodiment, the substrate of OATP1B1 is lovastatin. In an embodiment, the substrate of OATP1B1 is pitavastatin. In an embodiment, the substrate of OATP1B1 is simvastatin.

In another embodiment, the substrate of OATP1B1 is rosuvastatin. In some embodiments, the co-administered dose of rosuvastatin does not exceed 5 mg daily. In another embodiment, the co-administered dose of rosuvastatin is about 5 mg. In another embodiment, the clinically-recommended dose of rosuvastatin is about 5-40 mg, about 10-40 mg, about 10-20 mg, about 10-15 mg, about 5 mg, about 10 mg, about 15 mg, about 20 mg, about 30 mg, or about 40 mg. In another embodiment, the clinically-recommended dose of rosuvastatin is about 10 mg and the co-administered dose is reduced to about 5 mg. In another embodiment, the clinically-recommended dose of rosuvastatin is about 20 mg and the co-administered dose is reduced to about 10 mg or about 5 mg. In another embodiment, the clinically-recommended dose of rosuvastatin is about 30 mg and the co-administered dose is reduced to about 20 mg, about 10 mg or about 5 mg. In another embodiment, the clinically-recommended dose of rosuvastatin is about 5 mg and the co-administered dose is reduced to less than about 5 mg, such as about 4 mg, about 3 mg, about 2.5 mg, about 2 mg, or about 1 mg.

In some embodiments, a substrate of BCRP is co-administered to the subject with the Compound.

In another embodiment, the substrate of BCRP is at least one of 2-amino-1-methyl-6-phenylimidazo[4,5-b]pyridine (PhIP), estradiol-17-beta-glucuronide, estrone-3-sulfate, methotrexate, rosuvastatin, prazosin, or sulfasalazine. In some embodiments, the substrate of BCRP is a statin.

In another embodiment, the substrate of BCRP is rosuvastatin. In some embodiments, the co-administered dose of rosuvastatin does not exceed 5 mg daily. In another embodiment, the co-administered dose of rosuvastatin is about 5 mg. In another embodiment, the clinically-recommended dose of rosuvastatin is about 5-40 mg daily and the co-administered dose does not exceed 5 mg daily. In another embodiment, the clinically-recommended dose of rosuvastatin is about 10 mg and the co-administered dose is reduced to about 5 mg. In another embodiment, the clinically-recommended dose of rosuvastatin is about 5-40 mg, about 10-40 mg, about 10-20 mg, or 10-15 mg, about 5 mg, about 10 mg, about 15 mg, or about 20 mg. In another embodiment, the clinically-recommended dose of rosuvastatin is about 5 mg and the co-administered dose is reduced to less than about 5 mg, such as about 4 mg, about 3 mg, about 2.5 mg, about 2 mg, or about 1 mg.

In some embodiments, a substrate of OATP1B1 and BCRP is co-administered to the subject with the Compound. In an embodiment, the substrate of OATP1B1 and BCRP is a statin. In an embodiment, the substrate of OATP1B1 and BCRP is rosuvastatin.

In some embodiments, a substrate of P-gp is co-administered to the subject with the Compound.

In another embodiment, the substrate of P-gp is at least one of digoxin, fexofenadine, loperamide, N-methylquinidine (NMQ), quinidine, talinolol, vinblastine, or dabigatran etexilate.

In another embodiment, the substrate of P-gp is dabigatran etexilate. In some embodiments, the co-administered dose of dabigatran etexilate is less than about 300 mg, less than about 150 mg, or is less than about 75 mg. In an embodiment, the co-administered dose of dabigatran etexilate does not exceed about 75 mg daily. In an embodiment, the co-administered dose of dabigatran etexilate does not exceed about 150 mg daily. In another embodiment, the co-administered dose of dabigatran etexilate is about 75 mg. In another embodiment, the clinically-recommended dose of dabigatran etexilate is between about 75 mg and 300 mg and the co-administered dose is reduced to about 75 mg.

In another embodiment, more than one substrate of OATP1B1, BCRP, or P-gp is co-administered to the patient.

In an embodiment, a substrate of UGT1A1 is co-administered to the subject with the Compound. In an embodiment, the substrate of UGT1A1 is raltegravir.

In some embodiments, the Compound is administered once daily.

In some embodiments, the Compound is administered twice daily.

In some embodiments, the Compound is administered orally.

In another embodiment, the Compound comprises about 200 mg belumosudil mesylate salt, and the dose of the at least one substrate of OATP1B1, BRCP, or P-gp is reduced by about half as compared to the clinically-recommended dose when the substrate is administered without the Compound. In some embodiments, the at least one substrate of OATP1B1, BRCP, or P-gp is rosuvastatin, the dose of rosuvastatin does not exceed 5 mg daily, and the about 200 mg of belumosudil mesylate salt is administered once daily. In some embodiments, the at least one substrate of OATP1B1, BRCP, or P-gp is rosuvastatin, the dose of rosuvastatin does not exceed 5 mg daily, and the about 200 mg of belumosudil mesylate salt is administered twice daily. In some embodiments, the about 200 mg of belumosudil mesylate salt is expressed as the free base equivalent.

In some embodiments, the co-administered dose of rosuvastatin is about 10 mg. In some embodiments, the co-administered dose of rosuvastatin is about 5 mg. In some embodiments, the co-administered dose of rosuvastatin is less than about 5 mg, such as about 4 mg, about 3 mg, about 2.5 mg, about 2 mg, or about 1 mg.

In another embodiment, the dose of the Compound according to the co-administration disclosed herein is about 200 mg daily; in other embodiments, the dose of the Compound is about 400 mg daily; in other embodiments, the dose of the Compound is about 200 mg administered twice daily for a total daily dose of about 400 mg; in other embodiments, the dose of the Compound is 400 mg daily or 400 mg twice daily. In some embodiments, the belumosudil mesylate salt is expressed as the free base equivalent.

In an embodiment, about 200 mg of belumosudil mesylate salt expressed as the free base equivalent is administered once daily.

In an embodiment, about 200 mg of belumosudil mesylate salt expressed as the free base equivalent is administered twice daily.

In some embodiments, the use or methods comprise a treatment cycle wherein the at least one substrate of OATP1B1, BCRP, or P-gp is co-administered on days on which the subject does not receive the Compound; in some embodiments, the at least one substrate of OATP1B1, BCRP, or P-gp is administered prior to administration of the Compound; in some embodiments, the at least one substrate of OATP1B1, BCRP or P-gp is administered substantially at the same time as the Compound; in some embodiments, the at least one substrate of OATP1B1, BCRP or P-gp is administered on the same day as the Compound.

In some embodiments, the use or methods comprise a treatment cycle wherein the at least one substrate of OATP1B1, BCRP, or P-gp and the Compound are administered daily.

In some embodiments, the use or methods comprise a treatment cycle wherein the at least one substrate of OATP1B1, BCRP, or P-gp and the Compound are administered with food.

In another embodiment, the dose of the at least one substrate of OATP1B1, BCRP, or P-gp is reduced to mediate the increase in exposure of the co-administered substrate due to the co-administration of the Compound. In some embodiments, a substrate of OATP1B1 and BCRP is co-administered with the Compound, and the dose of the substrate of OATP1B1 and BCRP is reduced to mediate the increase in exposure of the substrate due to the co-administration of the Compound. In some embodiments, the dose of rosuvastatin is reduced to mediate the increase in exposure of the substrate due to the co-administration of the Compound. In some embodiments, the dose of dabigatran etexilate is reduced to mediate the increase in exposure of the substrate due to the co-administration of the Compound.

In some embodiments, there is provided a method of treating chronic graft-versus-host-disease (cGVHD) in a subject who is concurrently taking at least one substrate of OATP1B1, BCRP, or P-gp comprising administering to the subject in need thereof a therapeutically effective amount of belumosudil mesylate salt.

In some embodiments, there is provided a method for treating a subject with cGVHD comprising the steps of:
(a) determining whether the subject is in need of co-administration of at least one substrate of OATP1B1, BCRP, or P-gp;
(b) determining the clinically-recommended dose of the at least one substrate of OATP1B1, BCRP, or P-gp for the subject when the substrate is administered without co-administration of 2-{3-[4-(1H-indazol-5-ylamino)-2-quinazolinyl]phenoxy}-N-(propan-2-yl) acetamide or a pharmaceutically acceptable salt thereof (Compound);
(c) determining a lower dose of the at least one substrate of OATP1B1, BCRP, or P-gp to be administered to the subject to mediate the increase in exposure of the substrate due to the administration of the Compound; and
(d) co-administering the at least one substrate of OATP1B1, BCRP, or P-gp and the Compound.

In an embodiment, a method for treating a subject with cGVHD comprises the step of monitoring the subject for signs and symptoms of excessive exposure to the substrate of OATP1B1 and BCRP. In an embodiment, the signs and symptoms of excessive exposure comprises elevated exposure levels of rosuvastatin. In an embodiment, the signs and symptoms of excessive exposure comprises elevated exposure levels of belusmodudil.

In some embodiments, the subject has involvement of at least 4 organs. In some embodiments, the subject has involvement of at least 3 organs. In some embodiments, the subject has involvement of at least 2 organs.

In some embodiments, the subject has chronic graft-versus-host disease and has failed one to three prior lines of systemic therapy for the chronic graft-versus-host disease. In some embodiments, the subject has chronic graft-versus-host disease and has failed at least two prior lines of systemic therapy for the chronic graft-versus-host disease. In some embodiments, the subject has chronic graft-versus-host disease and has failed two to five prior lines of systemic therapy for the chronic graft-versus-host disease. In some embodiments, the subject has failed at least one, at least two, at least three, at least four, at least five, at least six, at least seven, at least eight, at least nine or at least ten prior lines of systemic therapy for the chronic graft versus-host disease.

In some embodiments, the subject experienced a complete response to last treatment for the graft-versus-host disease prior to belumosudil. In some embodiments, the subject experienced a partial response to last treatment for the graft-versus-host disease prior to belumosudil. In some embodiments, stable disease during the last treatment for the graft versus-host disease prior to belumosudil. In some embodiments, the prior lines of systemic therapy for the chronic graft-versus host disease have been discontinued.

In some embodiments, the prior lines of systemic therapy are selected from the group consisting of prednisone, tacrolimus, ECP, sirolimus, ibrutinib, ruxolitinib, MMF, rituximab, MTX, cyclosporine, imatinib, ixazomib, and ofatumumab.

Herein is also provided the use of 2-{3-[4-(1H-indazol-5-ylamino)-2-quinazolinyl]phenoxy}-N-(propan-2-yl) acetamide or a pharmaceutically acceptable salt thereof (Compound), in the manufacture of a medicament useful in treating the pathological conditions indicated herein, particularly chronic graft-versus-host disease (cGVHD), by co-administration with at least one substrate of organic anion transporting polypeptide 1B1 (OATP1B1) or a pharmaceutically acceptable salt thereof, breast cancer resistance protein (BCRP) or a pharmaceutically acceptable salt thereof, or P-glycoprotein (P-gp) or a pharmaceutically acceptable salt thereof. In some embodiments, the use of the Compound is for the manufacture of a medicament for treating cGVHD by co-administration with at least one substrate of organic anion transporting polypeptide 1B1 (OATP1B1) or a pharmaceutically acceptable salt thereof. In some embodiments, the use of the Compound is for the manufacture of a medicament for treating cGVHD by co-administration with at least one substrate of breast cancer resistance protein (BCRP) or a pharmaceutically acceptable salt thereof. In some embodiments, the use of the Compound is for the manufacture of a medicament for treating cGVHD by co-administration with at least one substrate of P-glycoprotein (P-gp) or a pharmaceutically acceptable salt thereof.

Herein is also provided the use of 2-{3-[4-(1H-indazol-5-ylamino)-2-quinazolinyl]phenoxy}-N-(propan-2-yl) acetamide or a pharmaceutically acceptable salt thereof (Compound), in the manufacture of medicament for treating chronic graft-versus-host disease (cGVHD), wherein the Compound is used in combination with at least one substrate of organic anion transporting polypeptide 1B1 (OATP1B1) or a pharmaceutically acceptable salt thereof, breast cancer resistance protein (BCRP) or a pharmaceutically acceptable salt thereof, or P-glycoprotein (P-gp) or a pharmaceutically acceptable salt thereof. In some embodiments, the Compound is used in combination with at least one substrate of organic anion transporting polypeptide 1B1 (OATP1B1) or a pharmaceutically acceptable salt thereof. In some embodiments, the Compound is used in combination with at least one substrate of breast cancer resistance protein (BCRP) or a pharmaceutically acceptable salt thereof. In some embodiments, the Compound is used in combination with at least one substrate of P-glycoprotein (P-gp) or a pharmaceutically acceptable salt thereof.

The present embodiments can be understood more fully by reference to the detailed description and examples, which are intended to exemplify non-limiting embodiments.

Embodiment 1. A method of treating chronic graft-versus-host-disease (cGVHD) in a subject in need thereof, the method comprising co-administering at least one substrate of organic anion transporting polypeptide 1B1 (OATP1B1), breast cancer resistance protein (BCRP), or P-glycoprotein (P-gp) and 2-{3-[4-(1H-indazol-5-ylamino)-2-quinazolinyl]phenoxy}-N-(propan-2-yl) acetamide or a pharmaceutically acceptable salt thereof (Compound), wherein the dose of the at least one substrate of OATP1B1, BCRP, or P-gp is reduced as compared to the clinically-recommended dose when the substrate is administered without the Compound.

Embodiment 2. The method according to Embodiment 1, wherein the Compound comprises the mesylate salt of 2-{3-[4-(1H-indazol-5-ylamino)-2-quinazolinyl]phenoxy}-N-(propan-2-yl) acetamide.

Embodiment 3. The method according to Embodiments 1 or 2, wherein the dose of the Compound is about 200 mg.

Embodiment 4. The method according to any one of Embodiments 1 to 3, wherein a substrate of OATP1B1 is co-administered to the subject.

Embodiment 5. The method according to Embodiment 4, wherein the substrate of OATP1B1 is at least one of estradiol-17β-glucuronide, pitavastatin, pravastatin, rosuvastatin, atorvastatin, lovastatin, or simvastatin.

Embodiment 6. The method according to Embodiment 4, wherein the substrate of OATP1B1 is a statin.

Embodiment 7. The method according to Embodiment 6, wherein the substrate of OATP1B1 is rosuvastatin.

Embodiment 8. The method according to Embodiment 7, wherein the co-administered dose of rosuvastatin does not exceed 5 mg daily.

Embodiment 9. The method according to Embodiment 7, wherein the co-administered dose of rosuvastatin is about 5 mg.

Embodiment 10. The method according to Embodiment 7, wherein the clinically-recommended dose of rosuvastatin is about 5-40 mg daily and the co-administered dose does not exceed about 5 mg daily.

Embodiment 11. The method according to Embodiment 7, wherein the clinically-recommended dose of rosuvastatin is about 10 mg and the co-administered dose does not exceed about 5 mg.

Embodiment 12. The method according to any one of Embodiments 1 to 3, wherein a substrate of BCRP is co-administered to the subject.

Embodiment 13. The method according to Embodiment 12, wherein the substrate of BCRP is at least one of 2-amino-1-methyl-6-phenylimidazo[4,5-b]pyridine (PhIP), estradiol-17-beta-glucuronide, estrone-3-sulfate, methotrexate, rosuvastatin, prazosin, or sulfasalazine.

Embodiment 14. The method according to Embodiment 12, wherein the substrate of BCRP is a statin.

Embodiment 15. The method according to Embodiment 12, wherein the substrate of BCRP is rosuvastatin.

Embodiment 16. The method according to Embodiment 12, wherein the substrate of BCRP is rosuvastatin calcium salt.

Embodiment 17. The method according to Embodiments 15 or 16, wherein the co-administered dose of rosuvastatin does not exceed 5 mg daily.

Embodiment 18. The method according to Embodiments 15 or 16, wherein the co-administered dose of rosuvastatin is about 5 mg.

Embodiment 19. The method according to Embodiments 15 or 16, wherein the clinically-recommended dose of rosuvastatin is about 5-40 mg daily and the co-administered dose does not exceed about 5 mg daily.

Embodiment 20. The method according to Embodiments 15 or 16, wherein the clinically-recommended dose of rosuvastatin is about 10 mg and the co-administered dose does not exceed about 5 mg.

Embodiment 21. The method according to any one of Embodiments 1-3, wherein a substrate of P-gp is co-administered to the subject.

Embodiment 22. The method according to Embodiment 21, wherein the substrate of P-gp is at least one of digoxin, fexofenadine, loperamide, N-methylquinidine (NMQ), quinidine, talinolol, vinblastine, or dabigatran.

Embodiment 23. The method according to Embodiment 21, wherein the substrate of P-gp is dabigatran.

Embodiment 24. The method according to Embodiment 21, wherein the substrate of P-gp is dabigatran etexilate.

Embodiment 25. The method according to Embodiment 24, wherein the co-administered dose of dabigatran etexilate is less than about 300 mg.

Embodiment 26. The method according to Embodiment 24, wherein the co-administered dose of dabigatran etexilate is less than about 150 mg.

Embodiment 27. The method according to Embodiment 24, wherein the co-administered dose of dabigatran etexilate is less than about 75 mg.

Embodiment 28. The method according to Embodiment 24, wherein the co-administered dose of dabigatran etexilate does not exceed 75 mg daily.

Embodiment 29. The method according to Embodiment 24, wherein the co-administered dose of dabigatran etexilate does not exceed 150 mg daily.

Embodiment 30. The method according to Embodiment 24, wherein the co-administered dose of dabigatran etexilate is about 75 mg.

Embodiment 31. The method according to Embodiment 24, wherein the clinically-recommended dose of dabigatran etexilate is between about 75 mg and 300 mg.

Embodiment 32. The method according to Embodiment 24, wherein the clinically-recommended dose of dabigatran etexilate is between about 150 mg and 300 mg and the co-administered dose does not exceed about 75 mg.

Embodiment 33. The method according to Embodiment 1, wherein more than one substrate of OATP1B1, BCRP, or P-gp is co-administered to the patient.

Embodiment 34. The method according to any one of Embodiments 1 to 33, wherein the Compound is administered once daily.

Embodiment 35. The method according to any one of Embodiments 1 to 33, wherein the Compound is administered twice daily.

Embodiment 36. The method according to Embodiment 1, wherein the dose of the Compound is about 200 mg, and wherein the dose of the at least one substrate of OATP1B1, BRCP, or P-gp is reduced by about half as compared to the clinically-recommended dose when the substrate is administered without the Compound.

Embodiment 37. The method according to Embodiment 36, wherein the at least one substrate of OATP1B1, BCRP, or P-gp is rosuvastatin.

Embodiment 38. The method according to Embodiment 37, wherein the daily dose of rosuvastatin does not exceed 5 mg of the free base form daily.

Embodiment 39. The method according to Embodiment 37, wherein the dose of rosuvastatin does not exceed 5 mg daily and the Compound is administered once daily.

Embodiment 40. The method according to Embodiment 37, wherein the dose of rosuvastatin does not exceed 5 mg daily and the Compound is administered twice daily.

Embodiment 41. The method according to Embodiment 37, wherein the co-administered dose of rosuvastatin is about 10 mg.

Embodiment 42. The method according to any one of Embodiments 36 to 41, wherein the Compound comprises belumosudil mesylate salt.

Embodiment 43. The method according to any one of Embodiments 1 to 42, comprising a treatment cycle wherein the at least one substrate of OATP1B1, BCRP, or P-gp is co-administered on days on which the subject does not receive the Compound.

Embodiment 44. The method according to any one of Embodiments 1 to 42, comprising a treatment cycle wherein the at least one substrate of OATP1B1, BCRP or P-gp is administered substantially at the same time as the Compound.

Embodiment 45. The method according to any one of Embodiments 1 to 42, comprising a treatment cycle wherein the at least one substrate of OATP1B1, BCRP or P-gp is administered on the same day as the Compound.

Embodiment 46. The method according to any one of Embodiments 1 to 42, comprising a treatment cycle wherein the at least one substrate of OATP1B1, BCRP or P-gp and the Compound are administered daily.

Embodiment 47. The method according to any one of Embodiments 1 to 42, comprising a treatment cycle wherein the at least one substrate of OATP1B1, BCRP or P-gp and the Compound are administered with food.

Embodiment 48. The method according to any one of Embodiments 1 to 47, wherein the subject has chronic graft-versus-host disease and has failed at least two prior lines of systemic therapy for the chronic graft-versus-host disease.

Embodiment 49. The method according to Embodiment 1, wherein the dose of the at least one substrate of OATP1B1, BCRP, or P-gp is reduced to mediate the increase in exposure of the co-administered substrate due to the co-administration of the Compound.

Embodiment 50. The method according to Embodiment 1, wherein the at least one substrate of OATP1B1, BCRP, or P-gp is a substrate of OATP1B1 and BCRP, and wherein the dose of the substrate of OATP1B1 and BCRP is reduced to mediate the increase in exposure of the substrate due to the co-administration of the Compound.

Embodiment 51. A method for treating a subject with cGVHD comprising the steps of: (a) determining whether the subject is in need of co-administration of at least one substrate of OATP1B1, BCRP, or P-gp; (b) determining the clinically-recommended dose of the at least one substrate of OATP1B1, BCRP, or P-gp for the subject when the substrate is administered without co-administration of 2-{3-[4-(1H-indazol-5-ylamino)-2-quinazolinyl]phenoxy}-N-(propan-2-yl) acetamide or a pharmaceutically acceptable salt thereof (Compound); (c) determining a lower dose of the at least one substrate of OATP1B1, BCRP, or P-gp to be administered to the subject to mediate the increase in exposure of the substrate due to the administration of the Compound; and (d) co-administering the at least one substrate of OATP1B1, BCRP, or P-gp and the Compound.

Embodiment 52. The method according to Embodiment 51, wherein the at least one substrate of OATP1B1, BCRP, or P-gp is a substrate of OATP1B1 and BCRP.

Embodiment 53. The method according to Embodiment 52, further comprising the step of monitoring the subject for signs and symptoms of excessive exposure to the substrate of OATP1B1 and BCRP.

Embodiment 54. The method according to any one of Embodiments 51-53, wherein the Compound is co-administered at a dosage of about 200 mg daily.

Embodiment 55. The method according to any one of Embodiments 51-53, wherein the Compound is co-administered at a dosage of about 400 mg daily.

Embodiment 56. The method according to any one of Embodiments 51-55, wherein the Compound comprises belumosudil mesylate salt.

Embodiment 57. The method according to Embodiment 51, wherein the at least one substrate of OATP1B1, BCRP, or P-gp is rosuvastatin.

Embodiment 58. The method according to Embodiment 51, wherein the at least one substrate of OATP1B1, BCRP, or P-gp is rosuvastatin calcium salt.

Embodiment 59. The method according to Embodiments 57 or 58, wherein the rosuvastatin is co-administered at a dose of about 5 mg daily.

Embodiment 60. The method according to Embodiments 57 or 58, wherein the rosuvastatin is co-administered at a dose not exceeding about 5 mg daily.

The following abbreviations may be helpful in considering the Examples and description herein.

### Abbreviations

| | |
|---|---|
| AE | Adverse events |
| alloHCT | allogeneic hematopoietic cell |
| | transplantation |
| BID | Twice daily (bi-daily) |
| BM | Bone marrow |
| cGVHD | Chronic graft versus host disease |
| CMV | cytomegalovirus |
| CR | Complete response |
| DDI | Drug-drug interaction |
| DOR | Duration of response |
| FFS | Failure-free survival |
| HLA | human leukocyte antigen |
| IST | Immunosuppressive therapy |
| LSS | Lee Symptom Scale |
| ORR | Overall response rate |
| OS | Overall survival |
| PBSC | peripheral blood stem cells |
| PPI | Proton pump inhibitors |
| PR | Partial response |
| QOL | Quality of life |
| SD | Standard deviation |
| SR | Steroid refractory |
| TEAEs | treatment-emergent adverse events |
| TTNT | Time to next treatment |
| QD | Daily; every day |

### EXAMPLES

### Example 1: United States REZUROCK™ (belumosudil) FDA Label

### Indications and Usage

REZUROCK is a kinase inhibitor indicated for the treatment of adult and pediatric patients 12 years and older with chronic graft-versus-host disease (chronic GVHD) after failure of at least two prior lines of systemic therapy. (1)

### Dosage and Administration

Recommended Dosage: 200 mg taken orally once daily with food. (2.1)

### Dosage Forms and Strengths

Tablet: 200 mg. (3)

### Contraindications

None. (4)

### Warnings and Precautions

Embryo-Fetal Toxicity: Can cause fetal harm. Advise females of reproductive potential of the potential risk to a fetus and to use effective contraception. (5.1, 8.1, 8.3)

### Drug Interactions

Strong CYP3A Inducers: Increase REZUROCK dosage to 200 mg twice daily. (7.1) Proton Pump Inhibitors: Increase REZUROCK dosage to 200 mg twice daily. (7.1)

### Adverse Reactions

The most common (≥20%) adverse reactions, including laboratory abnormalities, were infections, asthenia, nausea, diarrhea, dyspnea, cough, edema, hemorrhage, abdominal pain, musculoskeletal pain, headache, phosphate decreased, gamma glutamyl transferase increased, lymphocytes decreased, and hypertension. (6.1)

### Use in Specific Populations

Lactation: Advise not to breastfeed. (8.2)

See 17 for PATIENT COUNSELING INFORMATION and FDA-approved patient labeling.

### Full Prescribing Information

### 1 Indications and Usage

REZUROCK is indicated for the treatment of adult and pediatric patients 12 years and older with chronic graft-versus-host disease (chronic GVHD) after failure of at least two prior lines of systemic therapy.

### 2 Dosage and Administration

### 2.1 Recommended Dosage

The recommended dose of REZUROCK is 200 mg given orally once daily until progression of chronic GVHD that requires new systemic therapy.
Instruct the patient on the following:
Swallow REZUROCK tablets whole. Do not cut, crush, or chew tablets.
Take REZUROCK with a meal at approximately the same time each day [see Clinical Pharmacology (12.3)].
If a dose of REZUROCK is missed, instruct the patient to not take extra doses to make up the missed dose.

Treatment with REZUROCK has not been studied in patients with pre-existing severe renal or hepatic impairment. For patients with pre-existing severe renal or hepatic impairment, consider the risks and potential benefits before initiating treatment with REZUROCK [see Clinical Pharmacology (12.3)].

### 2.2 Dose Modifications for Adverse Reactions

Monitor total bilirubin, aspartate aminotransferase (AST), and alanine aminotransferase (ALT) at least monthly. Modify the REZUROCK dosage for adverse reactions as per Table 1.

TABLE 1
Recommended Dosage Modifications for
REZUROCK for Adverse Reactions

| Adverse | | REZUROCK |
|---|---|---|
| Reaction | Severity* | Dosage Modifications |
| Hepatotoxicity | Grade 3 AST or ALT | Hold REZUROCK until |
| [see Adverse | (5x to 20x ULN) or | recovery of bilirubin, |
| Reactions (6.1)] | Grade 2 bilirubin | AST and ALT to |
| | (1.5x to 3x ULN) | Grade 0-1, then resume |
| | | REZUROCK at the |
| | | recommended dose. |
| | Grade 4 AST or ALT | Discontinue REZUROCK |
| | (more than 20x ULN) or | permanently. |
| | Grade ≥ 3 bilirubin | |
| | (more than 3x ULN) | |
| Other adverse | Grade 3 | Hold REZUROCK until |
| reactions | | recovery to Grade 0-1, |
| [see Adverse | | then resume REZUROCK |
| Reactions (6.1)] | | at the recommended |
| | | dose level. |
| | Grade 4 | Discontinue REZUROCK |
| | | permanently. |

*Based on CTCAE v 4.03

### 2.3 Dosage Modification Due to Drug Interactions

### Strong CYP3A Inducers

Increase the dosage of REZUROCK to 200 mg twice daily when coadministered with strong CYP3A inducers

[see Drug Interactions (7.1)].

### Proton Pump Inhibitors

Increase the dosage of REZUROCK to 200 mg twice daily when coadministered with proton pump inhibitors [see Drug Interactions (72.1)].

### 3 Dosage Forms and Strengths

Each 200 mg tablet is a pale yellow film-coated oblong tablet debossed with “KDM” on one side and “200” on the other side.

### 4 Contraindications

None.

### 5 Warnings and Precautions

### 5.1 Embryo-Fetal Toxicity

Based on findings in animals and its mechanism of action, REZUROCK can cause fetal harm when administered to a pregnant woman. In animal reproduction studies, administration of belumosudil to pregnant rats and rabbits during the period organogenesis caused adverse developmental outcomes including embryo-fetal mortality and malformations at maternal exposures (AUC) less than those in patients at the recommended dose. Advise pregnant women of the potential risk to a fetus. Advise females of reproductive potential and males with female partners of reproductive potential to use effective contraception during treatment with REZUROCK and for at least one week after the last dose [see Use in Specific Populations (8.1, 8.3), Nonclinical Toxicology (13.1)].

### 6 Adverse Reactions

### 6.1 Clinical Trial Experience

Because clinical trials are conducted under widely variable conditions, adverse reaction rates observed in clinical trials of a drug cannot be directly compared with rates of clinical trials of another drug and may not reflect the rates observed in practice.

### Chronic Graft Versus Host Disease

In two clinical trials (Study KD025-213 and Study KD025-208), 83 adult patients with chronic GVHD were treated with REZUROCK 200 mg once daily [see Clinical Studies (14.1)]. The median duration of treatment was 9.2 months (range 0.5 to 44.7 months).

Fatal adverse reaction was reported in one patient with severe nausea, vomiting, diarrhea and multi-organ failure.

Permanent discontinuation of REZUROCK due to adverse reactions occurred in 18% of patients. The adverse reactions which resulted in permanent discontinuation of REZUROCK in >3% of patients included nausea (4%). Adverse reactions leading to dose interruption occurred in 29% of patients. The adverse reactions leading to dose interruption in ≥2% were infections (11%), diarrhea (4%), and asthenia, dyspnea, hemorrhage, hypotension, liver function test abnormal, nausea, pyrexia, edema, and renal failure with (2% each).

The most common (≥20%) adverse reactions, including laboratory abnormalities, were infections, asthenia, nausea, diarrhea, dyspnea, cough, edema, hemorrhage, abdominal pain, musculoskeletal pain, headache, phosphate decreased, gamma glutamyl transferase increased, lymphocytes decreased, and hypertension.

Table 2 summarizes the nonlaboratory adverse reactions.

TABLE 2
Nonlaboratory Adverse Reactions in ≥10% Patients
with Chronic GVHD Treated with REZUROCK

REZUROCK
200 mg once daily
(N = 83)

| | | |
|---|---|---|
| Adverse Reaction | All Grades (%) | Grades 3-4 (%) |

Infections and infestations

| | | |
|---|---|---|
| Infection (pathogen | 53 | 16 |
| not specified)^{a} | | |
| Viral infection^{b} | 19 | 4 |
| Bacterial infection^{c} | 16 | 4 |

General disorders and administration site conditions

| | | |
|---|---|---|
| Asthenia^{d} | 46 | 4 |
| Edema^{e} | 27 | 1 |
| Pyrexia | 18 | 1 |

Gastrointestinal

| | | |
|---|---|---|
| Nausea^{f} | 42 | 4 |
| Diarrhea | 35 | 5 |
| Abdominal pain^{g} | 22 | 1 |
| Dysphagia | 16 | 0 |

Respiratory, thoracic and mediastinal

| | | |
|---|---|---|
| Dyspnea^{h} | 33 | 5 |
| Coughⁱ | 30 | 0 |
| Nasal congestion | 12 | 0 |

Vascular

| | | |
|---|---|---|
| Hemorrhage^{j} | 23 | 5 |
| Hypertension | 21 | 7 |

Musculoskeletal and connective tissue

| | | |
|---|---|---|
| Musculoskeletal pain^{k} | 22 | 4 |
| Muscle spasm | 17 | 0 |
| Arthralgia | 15 | 2 |

Nervous system

| | | |
|---|---|---|
| Headache^{l} | 21 | 0 |

Metabolism and nutrition

| | | |
|---|---|---|
| Decreased appetite | 17 | 1 |

Skin and subcutaneous

| | | |
|---|---|---|
| Rash^{m} | 12 | 0 |
| Pruritusⁿ | 11 | 0 |

a infection with an unspecified pathogen includes acute sinusitis, device related infection, ear infection, folliculitis, gastroenteritis, gastrointestinal infection, hordeolum, infectious colitis, lung infection, skin infection, tooth infection, urinary tract infection, wound infection, upper respiratory tract infection, pneumonia, conjunctivitis, sinusitis, respiratory tract infection, bronchitis, sepsis, septic shock.
b includes influenza, rhinovirus infection, gastroenteritis viral, viral upper respiratory tract infection, bronchitis viral, Epstein-Barr viremia, Epstein-Barr virus infection, parainfluenzae virus infection, Varicella zoster virus infection, viral infection.
c includes cellulitis, Helicobacter infection, Staphylococcal bacteremia, catheter site cellulitis, Clostridium difficile colitis, Escherichia urinary tract infection, gastroenteritis Escherichia coli, Pseudomonas infection, urinary tract infection bacterial.
d includes fatigue, asthenia, malaise.
e includes edema peripheral, generalized edema, face edema, localized edema, edema.
f includes nausea, vomiting.
g includes abdominal pain, abdominal pain upper, abdominal pain lower.
h includes dyspnea, dyspnea exertional, apnea, orthopnea, sleep apnea syndrome.
i includes cough, productive cough.
j includes contusion, hematoma, epistaxis, increased tendency to bruise, conjunctival hemorrhage, hematochezia, mouth hemorrhage, catheter site hemorrhage, hematuria, hemothorax, purpura.
k includes pain in extremity, back pain, flank pain, limb discomfort, musculoskeletal chest pain, neck pain, musculoskeletal pain.
l includes headache, migraine.
m includes rash, rash maculo-papular, rash erythematous, rash generalized, dermatitis exfoliative.
n includes pruritus, pruritus generalized.

Table 3 summarizes the laboratory abnormalities in REZUROCK.

TABLE 3
Selected Laboratory Abnormalities in Patients
with Chronic GVHD Treated with REZUROCK

| | REZUROCK | | |
|---|---|---|---|
| | 200 mg once daily | | |
| | Grade 0-1 | Grade 2-4 | Grade 3-4 |
| | Baseline | Max Post | Max Post |
| Parameter | (N) | (%) | (%) |
| Chemistry | | | |
| Phosphate Decreased | 76 | 28 | 7 |
| Gamma Glutamyl | 47 | 21 | 11 |
| Transferase Increased | | | |
| Calcium Decreased | 82 | 12 | 1 |
| Alkaline Phosphatase | 80 | 9 | 0 |
| Increased | | | |
| Potassium Increased | 82 | 7 | 1 |
| Alanine Aminotransferase | 83 | 7 | 2 |
| Increased | | | |
| Creatinine Increased | 83 | 4 | 0 |
| Hematology | | | |
| Lymphocytes Decreased | 62 | 29 | 13 |
| Hemoglobin Decreased | 79 | 11 | 1 |
| Platelets Decreased | 82 | 10 | 5 |
| Neutrophil Count Decreased | 83 | 8 | 4 |

### 7 Drug Interactions

### 7.1 Effect of Other Drugs on REZUROCK

### Strong CYP3A Inducers

Coadministration of REZUROCK with strong CYP3A inducers decreases belumosudil exposure [see Clinical Pharmacology (12.3)], which may reduce the efficacy of REZUROCK. Increase the dosage of REZUROCK when coadministered with strong CYP3A inducers [see Dosage and Administration (2.3)].

### Proton Pump Inhibitors

Coadministration of REZUROCK with proton pump inhibitors decreases belumosudil exposure [see Clinical Pharmacology (12.3)], which may reduce the efficacy of REZUROCK. Increase the dosage of REZUROCK when coadministered with proton pump inhibitors [see Dosage and Administration (2.3)].

### 8 Use In Specific Populations

### 8.1 Pregnancy

### Risk Summary

Based on findings from animal studies and the mechanism of action [see Clinical Pharmacology (12.1)], REZUROCK can cause fetal harm when administered to pregnant women. There are no available human data on REZUROCK use in pregnant women to evaluate for a drug-associated risk. In animal reproduction studies, administration of belumosudil to pregnant rats and rabbits during the period of organogenesis resulted in adverse developmental outcomes, including alterations to growth, embryo-fetal mortality, and embryo-fetal malformations at maternal exposures (AUC) approximately ≥3-(rat) and ≥0.07 (rabbit) times the human exposure (AUC) at the recommended dose (see Animal Data). Advise pregnant women and females of reproductive potential of the potential risk to the fetus. In the U.S. general population, the estimated background risk of major birth defects and miscarriage in clinically recognized pregnancies is 2 to 4% and 15 to 20%, respectively.

### Data

### Animal Data

Embryo-fetal development studies were conducted in rats with administration of belumosudil to pregnant animals during the period of organogenesis at oral doses of 25, 50, 150, and 300 mg/kg/day in a pilot study and doses of 15, 50, and 150 mg/kg/day in a pivotal study. In the pilot study, maternal toxicity and embryo-fetal developmental effects were observed. Maternal toxicity (reduced body weight gain) occurred at 150 and 300 mg/kg/day doses. Increased post-implantation loss occurred at 50 and 300 mg/kg/day. Fetal-malformations were observed at ≥50 mg/kg/day and included absence of anus and tail, omphalocele, and dome shaped head. The exposure (AUC) at 50 mg/kg/day in rats is approximately 3 times the human exposure at the recommended dose of 200 mg.

In an embryo-fetal developmental study in rabbits, pregnant animals administered oral doses of belumosudil at 50, 125, and 225 mg/kg/day during the period of organogenesis resulted in maternal toxicity and embryo-fetal developmental effects. Maternal toxicity (body weight loss and mortality) was observed at doses ≥125 mg/kg/day. Embryo-fetal effects were observed at doses ≥50 mg/kg/day and included spontaneous abortion, increased post-implantation loss, decreased percentage of live fetuses, malformations, and decreased fetal body weight. Malformations included those in the tail (short), ribs (branched, fused or deformed), sternebrae (fused), and neural arches (fused, misaligned, and deformed). The exposure (AUC) at 50 mg/kg/day in rabbits is approximately 0.07 times the human exposure at the recommended dose of 200 mg.

### 8.2 Lactation

### Risk Summary

There are no data available on the presence of belumosudil or its metabolites in human milk or the effects on the breastfed child, or milk production. Because of the potential for serious adverse reactions from belumosudil in the breastfed child, advise lactating women not to breastfeed during treatment with REZUROCK and for at least one week after the last dose.

### 8.3 Females and Males of Reproductive Potential

REZUROCK can cause fetal harm when administered to a pregnant woman [see Use in Specific Populations (8.1)].

### Pregnancy Testing

Verify the pregnancy status of females of reproductive potential prior to initiating treatment with REZUROCK. Contraception

### Females

Advise females of reproductive potential to use effective contraception during treatment with REZUROCK and for at least one week after the last dose of REZUROCK. If this drug is used during pregnancy or if the patient becomes pregnant while taking this drug, the patient should be informed of the potential hazard to a fetus.

### Males

Advise males with female partners of reproductive potential to use effective contraception during treatment with REZUROCK and for at least one week after the last dose of REZUROCK.

### Infertility

### Females

Based on findings from rats, REZUROCK may impair female fertility. The effect on fertility is reversible [see Nonclinical Toxicology (13.1)].

### Males

Based on findings from rats and dogs, REZUROCK may impair male fertility. The effects on fertility are reversible [see Nonclinical Toxicology (13.1)].

### 8.4 Pediatric Use

The safety and effectiveness of REZUROCK have been established in pediatric patients 12 years and older. Use of REZUROCK in this age group is supported by evidence from adequate and well-controlled studies of REZUROCK in adults with additional population pharmacokinetic data demonstrating that age and body weight had no clinically meaningful effect on the pharmacokinetics of drug substance, that the exposure of drug substance is expected to be similar between adults and pediatric patients age 12 years and older, and that the course of disease is sufficiently similar in adult and pediatric patients to allow extrapolation of data in adults to pediatric patients.

The safety and effectiveness of REZUROCK in pediatric patients less than 12 years old have not been established.

### 8.5 Geriatric Use

Of the 186 patients with chronic GVHD in clinical studies of REZUROCK, 26% were 65 years and older. No clinically meaningful differences in safety or effectiveness of REZUROCK were observed in comparison to younger patients.

### 11 Description

Belumosudil is a kinase inhibitor. The active pharmaceutical ingredient is belumosudil mesylate with the molecular formula C27H28N6O5S and the molecular weight is 548.62 g/mol. The chemical name for belumosudil mesylate is 2-{3-[4-(1H-indazol-5-ylamino)-2-quinazolinyl]phenoxy}-N-(propan-2-yl) acetamide methanesulfonate (1:1). The chemical structure is as follows:

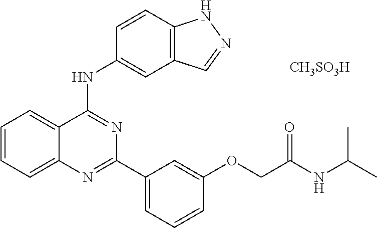

Belumosudil mesylate is a yellow powder that is practically insoluble in water, slightly soluble in methanol and DMF and soluble in DMSO.

REZUROCK tablets are for oral administration. Each tablet contains 200 mg of the free base equivalent to 242.5 mg of belumosudil mesylate. The tablet also contains the following inactive ingredients: microcrystalline cellulose, hypromellose, croscarmellose sodium, colloidal silicon dioxide, and magnesium stearate.

The tablet film consists of polyvinyl alcohol, polyethylene glycol, talc, titanium dioxide and yellow iron oxide.

### 12Clinical Pharmacology

### 12.1 Mechanism of Action

Belumosudil is an inhibitor of rho-associated, coiled-coil containing protein kinase (ROCK) which inhibits ROCK2 and ROCK1 with IC50 values of approximately 100 nM and 3 μM, respectively. Belumosudil down-regulated proinflammatory responses via regulation of STAT3/STAT5 phosphorylation and shifting Th17/Treg balance in ex-vivo or in vitro-human T cell assays. Belumosudil also inhibited aberrant pro-fibrotic signaling, in vitro. In vivo, belumosudil demonstrated activity in animal models of chronic GVHD.

### 12.2 Pharmacodynamics

Belumosudil exposure-response relationships and the time course of pharmacodynamic response are not established.

### 12.3 Pharmacokinetics

The following pharmacokinetic parameters are presented for chronic GVHD patients administered belumosudil 200 mg once daily, unless otherwise specified. The mean (% coefficient of variation, % CV) steady-state AUC and Cmax of belumosudil was 22700 (48%) h·ng/mL and 2390 (44%) ng/mL, respectively. Belumosudil Cmax and AUC increased in an approximately proportional manner over a dosage range of 200 and 400 mg (1 to 2 times once daily recommended dosage). The accumulation ratio of belumosudil was 1.4.

### Absorption

Median Tmax of belumosudil at steady state was 1.26 to 2.53 hours following administration of 200 mg once daily or twice daily in patients. The mean (% CV) bioavailability was 64% (17%) following a single belumosudil dose in healthy subjects.

### Effect of Food

Belumosudil Cmax and AUC increased 2.2 times and 2 times, respectively, following administration of a single belumosudil dose with a high-fat and high-calorie meal (800 to 1,000 calories with approximately 50% of total caloric content of the meal from fat) compared to the fasted state in healthy subjects. Median Tmax was delayed
0.5 hours.

### Distribution

The geometric mean volume of distribution after a single dose of belumosudil in healthy subjects was 184 L
(geo CV % 67.7%).

Belumosudil binding to human serum albumin and human □1-acid glycoprotein was 99.9% and 98.6%, respectively, in vitro.

### Elimination

The mean (% CV) elimination half-life of belumosudil was 19 hours (39%), and clearance was 9.83 L/hours (46%) in patients.

### Metabolism

Belumosudil is primarily metabolized by CYP3A4 and to a lesser extent by CYP2C8, CYP2D6, and UGT1A9, in vitro.

### Excretion

Following a single oral dose of radiolabeled belumosudil in healthy subjects, 85% of radioactivity was recovered in feces (30% as unchanged) and less than 5% in urine.

### Specific Populations

No clinically significant differences in belumosudil pharmacokinetics were observed with regard to age (18 to 77 years), sex, weight (38.6 to 143 kg), or mild to moderate renal impairment (eGFR≥60 and <90 mL/min/1.72 m² to eGFR≥30 and <60 mL/min/1.72 m²). The effect of severe renal impairment on the pharmacokinetics of belumosudil has not been studied.

### Drug Interaction Studies

### Clinical Studies and Model-Informed Approaches Effects of Other Drugs on Belumosudil

Strong Cytochrome P450 (CYP) 3A Inhibitors: There was no clinically meaningful effect on belumosudil exposure when coadministered with itraconazole in healthy subjects.

Strong CYP3A Inducers: Coadministration of rifampin decreased belumosudil Cmax by 59% and AUC by 72% in healthy subjects.

Moderate CYP3A Inducers: Coadministration of efavirenz is predicted to decrease belumosudil Cmax by 32% and AUC by 35% in healthy subjects.

Proton Pump Inhibitors: Coadministration of rabeprazole decreased belumosudil Cmax by 87% and AUC by 80%, and omeprazole decreased belumosudil Cmax by 68% and AUC by 47% in healthy subjects.

### Effects of Belumosudil on Other Drugs

CYP3A Substrates: Coadministration of belumosudil is predicted to increase midazolam (a sensitive CYP3A substrate) Cmax and AUC approximately 1.3- and 1.5-fold, respectively.

CYP2C9 Substrates: Coadministration of belumosudil is not expected to have clinically meaningful effect on the exposure of CYP2C9 substrates (such as warfarin).

CYP2C8 Substrates: Coadministration of belumosudil is not expected to have clinically meaningful effect on the exposure of CYP2C8 substrates that are not an OATP1B1 substrate.

### In Vitro Studies

Transporter Systems: Belumosudil is a substrate of P-gp. Belumosudil inhibits BCRP, P-gp, and OATP1B1 at clinically relevant concentrations.

Enzymes Systems: Belumosudil is an inhibitor of CYP1A2, CYP2C19, CYP2D6, UGT1A1 and UGT1A9.

### 13 Nonclinical Toxicology

### 13.1 Carcinogenesis, Mutagenesis, Impairment of Fertility

Carcinogenicity studies have not been conducted with belumosudil.

Belumosudil was not genotoxic in an in vitro bacterial mutagenicity (Ames) assay, in vitro chromosome aberration assay in human peripheral blood lymphocytes (HPBL) or an in vivo rat bone marrow micronucleus assay.

In a combined male and female rat fertility study, belumosudil-treated male animals were mated with untreated females, or untreated males were mated with belumosudil-treated females. Belumosudil was administered orally at doses of 50, 150 or 275 mg/kg/day to male rats 70 days prior to and throughout the mating period, and to female rats 14 days prior to mating and up to Gestation Day 7. At the dose of 275 mg/kg/day, adverse findings in female rats (treated with belumosudil or untreated but mated with treated males) included increased pre- or post-implantation loss and decreased number of viable embryos. Administration of belumosudil to male rats at a dose of 275 mg/kg/day resulted in abnormal sperm findings (reduced motility, reduced count, and increased percentage of abnormal sperm), and testes/epididymis organ changes (reduced weight and degeneration).

Fertility was reduced in both treated males or females at the 275 mg/kg/day dose and reached statistical significance in males. Adverse changes in male and female reproductive organs also occurred in general toxicology studies; findings included spermatozoa degeneration at a belumosudil dose of 35 mg/kg/day in dogs and decreased follicular development in ovaries at 275 mg/kg/day in rats. Changes were partially or fully reversed during the recovery period. The exposure (AUC) at the doses of 35 mg/kg/day in dogs, and 275 mg/kg/day in rats is 0.5 times and 8-9 times, respectively, the clinical exposure at the recommended dose of 200 mg daily.

### 14 Clinical Studies

### 14.1 Chronic Graft Versus Host Disease

Study KD025-213 (NCT03640481) was a randomized, open-label, multicenter study of REZUROCK for treatment of patients with chronic GVHD who had received 2 to 5 prior lines of systemic therapy and required additional treatment. Patients were excluded from the studies if platelets were <50×10⁹/L; absolute neutrophil count <1.5×10⁹/L; AST or ALT>3×ULN; total bilirubin>1.5×ULN; QTc(F)>480 ms; eGFR<30 mL/min/1.73 m²; or FEV1≤39%. There were 66 patients treated with REZUROCK 200 mg taken orally once daily. Concomitant treatment with supportive care therapies for chronic GVHD was permitted. Concomitant treatment with GVHD prophylaxis and standard care systemic chronic GVHD therapies was permitted as long as the subject has been on a stable dose for at least 2 weeks prior to study. Initiation of new systemic chronic GVHD therapy while on study was not permitted.

Demographics and baseline characteristics are summarized in Table 4.

TABLE 4
Demographics and Baseline Characteristics
of Patients with Chronic GVHD

| | REZUROCK |
|---|---|
| | 200 mg once daily |
| | (N = 65) |
| Age, Median, Years (minimum, | 53 |
| maximum) | (21, 77) |
| Age ≥65 Years, n (%) | 17 |
| | (26) |
| Male, n (%) | 42 |
| | (65) |
| Race, n (%) | |
| White | 54 |
| | (83) |
| Black | 6 |
| | (9) |
| Other or Not Reported | 5 |
| | (8) |
| Median (range) time (months) | 25.3 |
| from Chronic GVHD Diagnosis | (1.9, 162.4) |
| ≥4 Organs Involved, n (%) | 31 |
| | (48) |
| Median (range) Number of Prior | 3 |
| Lines of Therapy | (2, 6) |
| Number of Prior Lines of Therapy, n (%) | |
| 2 | 23 |
| | (35) |
| 3 | 12 |
| | (19) |
| 4 | 15 |
| | (23) |
| ≥5 | 15 |
| | (23) |
| Prior chronic GVHD treatment | 21 |
| with ibrutinib, n (%) | (32) |
| Prior chronic GVHD treatment | 20 |
| with ruxolitinib, n (%) | (31) |
| Refractory to Last Therapy, | 43/55 |
| n (%^{a}) | (78) |
| Severe chronic GVHD, n (%) | 46 |
| | (71) |
| Median (range) Global Severity | 7 |
| Rating | (2, 9) |
| Median (range) Lee Symptom | 27 |
| Scale Score at baseline | (7, 56) |
| Median (range) Corticosteroid | 0.19 |
| dose at baseline (PE/kg)^{b} | (0.03, 0.95) |

^{a}Denominator excludes patients with unknown status
^{b}Prednisone equivalents/kilogram

The efficacy of REZUROCK was based on overall response rate (ORR) through Cycle 7 Day 1 where overall response included complete response or partial response according to the 2014 NIH Response Criteria. The ORR results are presented in Table 5. The ORR was 75% (95% CI: 63, 85). The median duration of response, calculated from first response to progression, death, or new systemic therapies for chronic GVHD, was 1.9 months (95% CI: 1.2, 2.9). The median time to first response was 1.8 months (95% CI: 1.0, 1.9). In patients who achieved response, no death or new systemic therapy initiation occurred in 62% (95% CI: 46, 74) of patients for at least 12 months since response.

TABLE 5
Overall Response Rate through Cycle 7 Day 1 for
Patients with Chronic GVHD in Study KD025-213

| | REZUROCK |
|---|---|
| | 200 mg once daily |
| | (N = 65) |
| Overall Response Rate (ORR) | 49 (75%) |
| 95% Confidence Interval^{a} | (63%, 85%) |
| Complete Response | 4 (6%) |
| Partial Response | 45 (69%) |

^{a}Estimated using Clopper-Pearson method
ORR results were supported by exploratory analyses of patient-reported symptom bother which showed at least a 7-point decrease in the Lee Symptom Scale summary score through Cycle 7 Day 1 in 52% (95% CI: 40, 65) of patients.

### 16 How Supplied/Storage And Handling

REZUROCK 200 mg tablets are supplied as pale yellow film-coated oblong tablets containing 200 mg of belumosudil (equivalent to 242.5 mg belumosudil mesylate). Each tablet is debossed with “KDM” on one side and “200” on the other side and is packaged as follows: 200 mg tablets in 30 count bottle: NDC 79802-200-30

Store at room temperature, 20° C. to 25° C. (68° F. to 77° F.); excursions permitted from 15° C. and 30° C. (59° F. to 86° F.) [see USP Controlled Room Temperature].

Dispense to patient in original container only. Store in original container to protect from moisture. Replace cap securely each time after opening. Do not discard desiccant.

### 17 Patient Counseling Information

Advise the patient to read the FDA-approved patient labeling (Patient Information).

### Embryo-Fetal Toxicity:

Advise pregnant women and females of reproductive potential of the potential risk to a fetus. Advise females of reproductive potential to inform their healthcare provider of a known or suspected pregnancy

[see Warnings and Precautions (5.1), Use in Specific Populations (8.1, 8.3)].
Advise females of reproductive potential to use effective contraceptive during treatment with REZUROCK and for at least one week after the last dose [see Warnings and Precautions (5.1)].
Advise males with female partners of reproductive potential to use effective contraceptive during treatment with REZUROCK and for at least one week after the last dose [see Use in Specific Populations (8.3)].

### Lactation

Advise women not to breastfeed during treatment with REZUROCK and for at least one week after the last dose [see Use in Specific Populations (8.2)].

### Infertility

Advise males and females of reproductive potential that REZUROCK may impair fertility [see Use in Specific Populations (8.3)].

### Administration

Inform patients to take REZUROCK orally once daily with food according to their physician's instructions and that the oral dosage (tablets) should be swallowed whole with a glass of water without cutting, crushing or chewing the tablets approximately the same time each day [see Dosage and Administration (2.1)].

Advise patients that in the event of a missed daily dose of REZUROCK, it should be taken as soon as possible on the same day with a return to the normal schedule the following day. Patients should not take extra doses to make up the missed dose [see Dosage and Administration (2.1)].

### Drug Interactions

Advise patients to inform their health care providers of all concomitant medications, including prescription medicines, over-the-counter drugs, vitamins, and herbal products [see Drug Interactions (7)].

TABLE 6
Patient Information Patient INFORMATION REZUROCK
(REZ-ur-ok) (belumosudil) tablets

What is REZUROCK?
REZUROCK is a prescription medicine used to treat adults and children 12 years of age and
older with chronic graft- versus-host disease (chronic GVHD) after you have received at least
2 prior treatments (systemic therapy) and they did not work.
It is not known if REZUROCK is safe and effective in children less than 12 years old.
Before taking REZUROCK, tell your healthcare provider about all of your medical
conditions, including if you:
have kidney or liver problems.
are pregnant or plan to become pregnant. REZUROCK can harm your unborn baby. If you
are able to become pregnant, your healthcare provider will do a pregnancy test before
starting treatment with REZUROCK. Tell your healthcare provider if you become pregnant
or think you may be pregnant during treatment with REZUROCK.
Females who can become pregnant should use effective birth control during treatment
with REZUROCK and for at least 1 week after the last dose.
Males with female partners who can become pregnant should use effective birth
control during treatment with REZUROCK and for at least 1 week after the last dose.
are breastfeeding or plan to breastfeed. It is not known if REZUROCK passes into breast
milk. Do not breastfeed during treatment with REZUROCK and for at least 1 week after
the last dose.
Tell your healthcare provider about all the medicines you take, including prescription and
over-the-counter medicines, vitamins, and herbal supplements. REZUROCK may affect the
way other medicines work, and other medicines may affect the way REZUROCK works.
Know the medicines you take. Keep a list of them to show your healthcare provider and
pharmacist when you get a new medicine.
How should I take REZUROCK?
Take REZUROCK exactly as your healthcare provider tells you to take it.
Do not change your dose or stop taking REZUROCK without first talking to your
healthcare provider.
Take REZUROCK 1 time a day with a meal.
Take REZUROCK at about the same time each day.
Swallow REZUROCK tablets whole with a glass of water.
Do not cut, crush, or chew REZUROCK tablets.
Your healthcare provider will do blood tests to check your liver at least 1 time a month
during treatment with REZUROCK.
If you miss a dose of REZUROCK, take it as soon as you remember on the same day.
Take your next dose of REZUROCK at your regular time on the next day. Do not take
extra doses of REZUROCK to make up for a missed dose.
If you take too much REZUROCK, call your healthcare provider or go to the nearest
hospital emergency room right away.
What are the possible side effects of REZUROCK?
The most common side effects of REZUROCK include:
infections □ swelling
tiredness or weakness □ bleeding
nausea □ stomach (abdominal) pain
diarrhea □ muscle or bone pain
shortness of breath □ headache
cough □ high blood pressure
Your healthcare provider may change your dose of REZUROCK, temporarily stop, or
permanently stop treatment with REZUROCK if you have certain side effects.
REZUROCK may affect fertility in males and females. Talk to your healthcare provider if this
is a concern for you. These are not all the possible side effects of REZUROCK.
Call your doctor for medical advice about side effects. You may report side effects to FDA
at 1-800-FDA-1088. You may also report side effects to Kadmon Pharmaceuticals, LLC at
1-877-377-7862.
How should I store REZUROCK?
Store REZUROCK at room temperature between 68° F. to 77° F. (20° C. to 25° C.).
Keep REZUROCK in its original container. The REZUROCK bottle contains a desiccant
packet to help keep your tablets dry (protect from moisture). Keep the desiccant in the
bottle.
Tightly close the REZUROCK bottle after you take your dose.
Keep REZUROCK and all medicines out of the reach of children.
General information about the safe and effective use of REZUROCK.
Medicines are sometimes prescribed for purposes other than those listed in a Patient
Information leaflet. Do not use REZUROCK for a condition for which it was not prescribed.
Do not give REZUROCK to other people, even if they have
the same symptoms that you have. It may harm them. You can ask your pharmacist or
healthcare provider for information about REZUROCK that is written for health professionals.
What are the ingredients in REZUROCK? Active ingredient: belumosudil mesylate
Inactive ingredients:
Tablet core: microcrystalline cellulose, hypromellose, croscarmellose sodium, colloidal silicon
dioxide, and magnesium stearate.
Tablet coating: polyvinyl alcohol, polyethylene glycol, talc, titanium dioxide and yellow iron
oxide.

### Example 2: Belusmosudil Interactions with Transport Protein Substrates

### Interactions

### Effect Of Other Drugs On Belumosudil

### Strong CYP3A4 Inducers

Coadministration of belumosudil with strong CYP3A inducers decreases belumosudil exposure, which may reduce the efficacy of belumosudil. Increase the dosage of belumosudil when co-administered with strong CYP3A inducers.

### Proton Pump Inhibitors

Coadministration of belumosudil with proton pump inhibitors decreases belumosudil exposure, which may reduce the efficacy of belumosudil. Increase the dosage of belumosudil when co-administered with proton pump inhibitors.

### Effect of Belumosudil on Other Drugs

### OATP1B1/BCRP Substrates:

Coadministration of belumosudil with drugs transported by OATP1B1 and BCRP can lead to an increase in exposure of these concomitant drugs (e.g. rosuvastatin) which may increase the risk of these substrate-related toxicities. Consider switching to a drug less sensitive to OATP1B1 and BCRP inhibition when possible. If used together the dose of rosuvastatin should not exceed 5 mg once daily. Monitor patients closely for signs and symptoms of excessive exposure to the drugs that are substrates of OATP1B1 and BCRP.

### Drug Interaction Studies

### Effects of Belumosudil on Other Drugs

### Transporter Systems

OATP1B1/BCRP substrates: Coadministration of belumosudil increases rosuvastatin Cmax and AUC by 3.6 and 4.6-fold, respectively.

P-glycoprotein (P-gp) substrates: Coadministration of belumosudil increased the exposure of dabigatran by 2-fold, indicating a moderate potential for inhibiting transport of P-gp substrates.

### Example 3: A Three-Part, Sequential, Non-Randomized, Open-Label Study Designed to Evaluate the Effect of Oral Belumosudil on UGT1A1, P-Gp, BCRP and OATP1B1 Inhibition in the Fed State in Healthy Male Subjects

### Objectives: Primary:

Part 1: To determine the effect of multiple doses of REZUROCK™ (belumosudil) tablet on the single dose oral PK of the UGT1A1 victim drug, ISENTRESS® (raltegravir).

Part 2: To determine the effect of multiple doses of REZUROCK (belumosudil) tablet on the single dose oral PK of the P-gp victim drug, PRADAXA® (dabigatran etexilate).

Part 3: To determine the effect of multiple doses of REZUROCK (belumosudil) tablet on the single dose oral PK of the OATP1B1/BCRP victim drug, CRESTOR® (rosuvastatin calcium).

### Secondary:

Part 1: To provide single dose PK data on the victim drug raltegravir and its metabolite, raltegravir glucuronide, following administration of raltegravir alone and in combination with REZUROCK (belumosudil) tablet.

Part 2: To provide single dose PK data on the victim drug dabigatran etexilate, alone and in combination with REZUROCK (belumosudil) tablet.

Part 3: To provide single dose PK data on the victim drug rosuvastatin, alone and in combination with REZUROCK (belumosudil) tablet.

Parts 1, 2, and 3: To determine the PK of belumosudil and known metabolites (KD025m1, KD025m2) following single and multiple oral doses of REZUROCK (belumosudil) tablet in healthy participants in the fed state.

Parts 1, 2, and 3: To provide additional safety and tolerability information for REZUROCK (belumosudil) tablet when coadministered with the UGT1A1 victim drug, raltegravir (Part 1), the P-gp victim drug, dabigatran etexilate (Part 2), and the OATP1B1/BCRP victim drug, rosuvastatin (Part 3).

### Methodology.

Part 1 was a single-center, nonrandomized, open-label, single-period design to assess the effect of multiple doses of belumosudil on the single-dose PK of raltegravir (a UGT1A1-sensitive substrate), and 19 healthy male participants were to be enrolled.

Part 2 was a single-center, nonrandomized, open-label, single-period design to assess the effect of multiple doses of belumosudil on the single-dose PK of dabigatran etexilate (a P-gp-sensitive substrate), and 19 healthy male participants were to be enrolled.

Part 3 was a single-center, nonrandomized, open-label, single-period design to assess the effect of multiple doses of belumosudil on the single-dose PK of rosuvastatin (a BCRP/OATP1B1-sensitive substrate), and 15 healthy male participants were to be enrolled.

Throughout this clinical study report, the term “study intervention” refers to the following:
Perpetrator investigational medicinal product (IMP): REZUROCK (belumosudil) tablets, 200 mg
Victim IMPs: ISENTRESS (raltegravir) film-coated tablets (400 mg), PRADAXA (dabigatran etexilate) capsules (75 mg), and CRESTOR (rosuvastatin calcium) tablets (10 mg)

### Parts 1, 2, and 3 Study Design

In each study part, participants underwent preliminary screening procedures for the study at the screening visit (Days −28 to −2 of each study part). Participants were admitted to the clinical unit on the morning of the day prior to first dose of study intervention (ie, on Day −1).

Enrolled participants were dosed with perpetrator and victim IMPs as follows:
Part 1 (Regimen A [raltegravir+belumosudil]): Participants received a single dose of the victim IMP raltegravir on the morning of Days 1 and 7. Additionally, participants received a single dose of the perpetrator belumosudil tablet on the morning of Days 3 to 8 (once daily [QD]). On Day 7, the victim and perpetrator IMPs were administered simultaneously.
Part 2 (Regimen B [dabigatran etexilate+belumosudil]): Participants received a single dose of the victim IMP dabigatran etexilate on the morning of Days 1 and 9. Additionally, participants received a single dose of the perpetrator belumosudil tablet on the morning of Days 5 to 12 (QD). On Day 9, the victim and perpetrator IMPs were administered simultaneously.
Part 3 (Regimen C [rosuvastatin calcium+belumosudil]): Participants received a single dose of the victim IMP rosuvastatin on the morning of Days 1 and 10. Additionally, participants received a single dose of the perpetrator belumosudil tablet on the morning of Days 6 to 13 (QD). On Day 10, the victim and perpetrator IMPs were administered simultaneously.

On the evening before each dose, participants were provided with a light snack and were required to fast from all food and drink (except water) for a minimum of 10 hours until the following morning. All study interventions were administered in the fed state, following completion of a standard breakfast (with dosing planned to occur 30 minutes after the start of the breakfast).

In each study part, blood samples were collected at regular intervals for the PK analysis of plasma concentrations of the following analytes: raltegravir and raltegravir glucuronide (Part 1); unconjugated dabigatran (Part 2); rosuvastatin (Part 3); and belumosudil and known metabolites (KD025m1, KD025m2) (Parts 1, 2, and 3). The following safety assessments were also performed in Parts 1, 2, and 3: incidence of adverse events (AEs), vital signs, electrocardiograms (ECGs), physical examinations, and laboratory safety tests.

All participants remained on site until 48 hours postfinal dose for safety and PK assessments, and a follow-up phone call was to take place at 3 to 7 days postfinal dose to ensure the ongoing wellbeing of the participants.

### Number of Participants.

Part 1 (Regimen A): It was planned to enroll 19 participants in Part 1. 19 participants were enrolled and were administered Regimen A (raltegravir+belumosudil), and all enrolled participants completed the study. All 19 enrolled participants were included in the safety and PK populations and analysis sets.

Part 2 (Regimen B): It was planned to enroll 19 participants in Part 2. 19 participants were enrolled and were administered Regimen B (dabigatran etexilate+belumosudil), and all enrolled participants completed the study. All 19 enrolled participants were included in the safety and PK populations and analysis sets.

Part 3 (Regimen C): It was planned to enroll 15 participants in Part 3; however, 1 participant was determined to be ineligible to dose on Day 1. Therefore, 14 participants were enrolled and were administered Regimen C (rosuvastatin calcium+belumosudil), and all enrolled participants completed the study. All 14 enrolled participants were included in the safety and PK populations and analysis sets.

### Diagnosis and Criteria for Inclusion.

This study enrolled healthy male participants aged 18 to 55 years inclusive, at time of signing informed consent, with a body mass index (BMI) 18.0 to 32.0 kg/m² and body weight ≥50 kg as measured at screening. In Part 1, participants identified to be poor metabolizers for UGT1A1 and in Part 3, participants identified to carry reduced or predicted reduced function genes for SLCO1B1 (OATP1B1) were excluded from the study.

### Study Products

Part 1 (Regimen A): Victim IMP: ISENTRESS (raltegravir) film coated tablets, for oral use, 400 mg (expressed as the free phenol equivalent)

Part 2 (Regimen B): Victim IMP: PRADAXA (dabigatran etexilate) capsules, for oral use, 75 mg (expressed as the free drug equivalent)

Part 3 (Regimen C): Victim IMP: CRESTOR (rosuvastatin calcium) tablets, 10 mg (expressed as the free drug equivalent)

Parts 1, 2, and 3 (Regimens A, B, and C, respectively): Perpetrator IMP: REZUROCK (belumosudil) tablet, 200 mg (expressed as the free base equivalent)

All study interventions were administered orally in the fed state.

### Duration of Study Intervention.

In Part 1, participants received a single dose of raltegravir on 2 separate occasions (morning of Days 1 and 7), and belumosudil QD for 6 consecutive days (morning of Days 3 to 8). In Part 2, participants received a single dose of dabigatran etexilate on 2 separate occasions (morning of Days 1 and 9), and belumosudil QD for 8 consecutive days (morning of Days 5 to 12). In Part 3, participants received a single dose of rosuvastatin on 2 separate occasions (morning of Days 1 and 10), and belumosudil QD for 8 consecutive days (morning of Days 6 to 13).

### Criteria for Evaluation.

### Primary.

Part 1: PK parameters: AUC(0-last) and AUC(0-inf) for raltegravir, as applicable, following administration of the test (raltegravir+belumosudil) and reference (raltegravir alone) treatments. Results of the formal statistical analysis of the PK parameters AUC(0-last) and AUC(0-inf) for raltegravir in the test treatment (raltegravir+belumosudil) compared to the reference treatment (raltegravir alone), where possible and appropriate.

Part 2: PK parameters: AUC(0-last) and AUC(0-inf) for unconjugated dabigatran, as applicable, following administration of the test (dabigatran etexilate+belumosudil) and reference (dabigatran etexilate alone) treatments. Results of the formal statistical analysis of the PK parameters AUC(0-last) and AUC(0-inf) for unconjugated dabigatran in the test treatment (dabigatran etexilate+belumosudil) compared to the reference treatment (dabigatran etexilate alone), where possible and appropriate.

Part 3: PK parameters: AUC(0-last) and AUC(0-inf) for rosuvastatin, as applicable, following administration of the test (rosuvastatin+belumosudil) and reference (rosuvastatin alone) treatments. Results of the formal statistical analysis of the PK parameters AUC(0-last) and AUC(0-inf) for rosuvastatin in the test treatment (rosuvastatin+belumosudil) compared to the reference treatment (rosuvastatin alone), where possible and appropriate.

### Secondary.

Part 1: PK parameters: Tmax, Cmax, and T_{1/2} for raltegravir, as applicable, following administration of the test (raltegravir+belumosudil) and reference (raltegravir alone) treatments. PK parameters: AUC(0-last), AUC(0-inf), Tmax, Cmax, and T_{1/2} for raltegravir glucuronide, as applicable, following administration of the test (raltegravir+belumosudil) and reference (raltegravir alone) treatments. Results of the formal statistical analysis of the PK parameter Cmax for raltegravir in the test treatment (raltegravir+belumosudil) compared to the reference treatment (raltegravir alone), where possible and appropriate. Results of the formal statistical analysis of the PK parameters Cmax, AUC(0-last), and AUC(0-inf) for raltegravir glucuronide in the test treatment (raltegravir+belumosudil) compared to the reference treatment (raltegravir alone), where possible and appropriate.

Part 2: PK parameters: Tmax, Cmax, and T_{1/2} for unconjugated dabigatran, as applicable, following administration of the test (dabigatran etexilate+belumosudil) and reference (dabigatran etexilate alone) treatments. Results of the formal statistical analysis of the PK parameter Cmax for unconjugated dabigatran in the test treatment (dabigatran etexilate+belumosudil) compared to the reference treatment (dabigatran etexilate alone), where possible and appropriate.

Part 3: PK parameters: Tmax, Cmax, and T_{1/2} for rosuvastatin, as applicable, following administration of the test (rosuvastatin+belumosudil) and reference (rosuvastatin alone) treatments. Results of the formal statistical analysis of the PK parameter Cmax for rosuvastatin in the test treatment (rosuvastatin+belumosudil) compared to the reference treatment (rosuvastatin alone), where possible and appropriate.

Parts 1, 2, and 3: PK parameters: Tmax, Cmax, AUC(0-last), AUC(0-tau), and T_{1/2}, for belumosudil, KD025m1, and KD025m2 as applicable.

Parts 1, 2, and 3: To provide additional safety and tolerability information for belumosudil by assessing: AEs, vital signs, ECGs, physical examinations, and laboratory safety tests following administration of the 3 victim drugs (raltegravir [Part 1], dabigatran etexilate [Part 2] and rosuvastatin [Part 3]) alone and in combination with belumosudil.

### Statistical Methods.

Formal statistical analysis was performed on the PK parameters maximum observed concentration (Cmax) and area under the curve from time 0 to the time of last measurable concentration (AUC(0-last)) for Regimens A, B, and C (Parts 1, 2 and 3), and area under the curve from time 0 extrapolated to infinity (AUC(0-inf)) for Regimen B (Part 2) only, separately for each regimen/part and included the following pairwise treatment comparisons (test versus reference, ie, victim+perpetrator IMPs versus victim IMP alone): Regimen A (Part 1): raltegravir+belumosudil (Day 7) versus raltegravir alone (Day 1) for parent raltegravir and its metabolite raltegravir glucuronide. Regimen B (Part 2): dabigatran+belumosudil (Day 9) versus dabigatran alone (Day 1) for unconjugated dabigatran. Regimen C (Part 3): rosuvastatin+belumosudil (Day 10) versus rosuvastatin alone (Day 1) for rosuvastatin. The PK parameters underwent a natural logarithmic transformation and were analyzed using a mixed effect modelling technique. Adjusted geometric mean ratios and 90% confidence intervals (CIs) of the ratios were obtained for each comparison. The ratio was defined as test/reference. In addition, the intraparticipant variability were also presented. Additional PK parameters were estimated for plasma raltegravir, raltegravir glucuronide, unconjugated dabigatran, and rosuvastatin, belumosudil and known metabolites (KD025m1 and KD025m2) where possible and appropriate. No formal statistical analyses were performed for the PK data for plasma belumosudil and known metabolites (KD025m1 and KD025m2), or for the safety data during this study. No formal interim analyses were performed for this study.

### Summary Results.

### Demographic and Other Baseline Characteristics.

Overall, 52 healthy male participants between 19 and 54 years of age inclusive were entered into the study; all had a body weight of at least 50 kg and were within the reference range for BMI (18.0 to 32.0 kg/m²) as required by the protocol. No participants were current smokers and all had an alcohol consumption of between 0 and 2 units per week.

### Exposure.

For Regimen A, all 19 participants received the planned total of 2 oral doses of raltegravir and 6 oral doses of belumosudil over 8 days: single doses of 400 mg raltegravir on Days 1 and 7, and QD doses of 200 mg belumosudil on Days 3 to 8. The cumulative dose of belumosudil received by each participant was 1200 mg and duration of exposure was 6 days.

For Regimen B, all 19 participants received the planned total of 2 oral doses of dabigatran etexilate and 8 oral doses of belumosudil over 12 days: single doses of 75 mg dabigatran etexilate on Days 1 and 9, and QD doses of 200 mg belumosudil on Days 5 to 12. The cumulative dose of belumosudil received by each participant was 1600 mg and duration of exposure was 8 days.

Although 15 participants were planned to be administered Regimen C, 1 participant was determined to be ineligible to dose on Part 3 Day 1. All 14 participants received the planned total of 2 oral doses of rosuvastatin calcium and 8 oral doses of belumosudil over 13 days: single doses of 10 mg rosuvastatin calcium on Days 1 and 10, and QD doses of 200 mg belumosudil on Days 6 to 13. The cumulative dose of belumosudil received by each participant was 1600 mg and duration of exposure was 8 days.

### Pharmacokinetic Results.

The statistical analysis results for the assessment of the effect of multiple doses of belumosudil on the single dose oral PK endpoints AUC(0-last), AUC(0-inf) (Regimen B [dabigatran etexilate+belumosudil; Part 2] only) and Cmax for raltegravir, raltegravir glucuronide, unconjugated dabigatran and rosuvastatin are presented in Table 7, below.

TABLE 7

| | | Victim IMP + | | Victim IMP | | | | |
|---|---|---|---|---|---|---|---|---|
| | | belumosudil | | alone | | | | |
| Regimen | | | Adj | | Adj | | | |
| (Part)/ | | | geo | | geo | Ratio | 90% Cl | CVw |
| analyte | Parameter | n | mean^{a} | n | mean^{a} | (%)^{b} | (%)^{c} | (%)^{d} |
| Regimen A | AUC₍₀₋ₗₐₛₜ₎ | 18 | 1500 | 18 | 1570 | 95.23 | (65.99, | 70.1 |
| (Part 1)/ | (ng · h/mL) | | | | | | 137.42) | |
| raltegravir | Cₘₐₓ | 18 | 447 | 18 | 512 | 87.28 | (60.80, | 68.9 |
| | (ng/mL) | | | | | | 125.29) | |
| Regimen A | AUC₍₀₋ₗₐₛₜ₎ | 19 | 859 | 19 | 1420 | 60.38 | (42.87, | 66.9 |
| (Part 1)/ | (ng · h/mL) | | | | | | 85.03) | |
| raltegravir | Cₘₐₓ | 19 | 207 | 19 | 359 | 57.58 | (42.51, | 58.1 |
| glucuronide | (ng/mL) | | | | | | 78.00) | |
| Regimen B | AUC₍₀₋ₗₐₛₜ₎ | 19 | 682 | 19 | 317 | 215.41 | (167.03, | 47.6 |
| (Part 2)/ | (ng · h/mL) | | | | | | 277.81) | |
| unconjugated | AUC(0-inf) | 16 | 822 | 16 | 398 | 206.47 | (160.20, | 42.7 |
| dabigatran | (ng · h/mL) | | | | | | 266.11) | |
| | Cₘₐₓ | 19 | 99.5 | 19 | 42.1 | 236.06 | (178.61, | 52.8 |
| | (ng/mL) | | | | | | 311.98) | |
| Regimen C | AUC₍₀₋ₗₐₛₜ₎ | 10 | 62.6 | 10 | 13.6 | 461.59 | (332.25, | 41.8 |
| (Part 3)/ | (ng · h/mL) | | | | | | 641.26) | |
| rosuvastatin | Cₘₐₓ | 11 | 10.6 | 11 | 2.96 | 359.21 | (231.34, | 61.9 |
| | (ng/mL) | | | | | | 557.75) | |

AUC(0-inf): area under the curve from time 0 extrapolated to infinity; AUC(0-last); area under the curve from time 0 to the time of last measurable concentration. Cmax: maximum observed concentration; Cl: confidence interval; IMP: investigational medicinal product; n: number of participants with an observation.
Victim investigation medicinal products = Regimen A (Part 1): raltegravir 400 mg single dose (Days 1 and 7); Regimen B (Part 2): dabigatran etexilate 75 mg single dose (Days 1 and 9); Regimen C (Part 3): rosuvastatin calcium 10 mg single dose (Days 1 and 10).
Perpetrator investigational medicinal products = belumosudil 200 mg QD, Days 3 to 8 for Regimen A (Part 1), Days 5 to 12 for Regimen B (Part 2), and Days 6 to 13 for Regimen C (Part 3).
Results obtained from mixed modelling of natural log transformed PK parameters with terms for treatment as a fixed effect and participant as a random effect. Only participants who have evaluable parameter estimates for both treatments have been included. AUC(0-inf) is excluded where there are insufficient evaluable estimates.
^{a}Adj geo mean = adjusted geometric mean.
^{b}Ratio of adj geo means (test/reference ie, victim + perpetrator/victim alone).
^{c}Confidence intervals for ratio of adj geo means.
^{d}CVw = intraparticipant variability.

For Regimen A (Part 1), geometric mean (geometric coefficient of variation [CV %]) values for plasma PK parameters for raltegravir and raltegravir glucuronide are presented in Table 8, below.

TABLE 8

| Treatment | | | Raltegravir + | |
|---|---|---|---|---|
| (Day) | Raltegravir alone | | belumosudil | |
| Number of | (Day 1) | | (Day 7) | |
| participants | N = 19 | | N = 19 | |
| Analyte | | Raltegravir | | Raltegravir |
| Parameter | Raltegravir | glucuronide | Raltegravir | glucuronide |
| Tₘₐₓ^{a} (h) | 3.000 | 4.000 | 3.000 | 4.000 |
| | (1.00-12.00) | (1.00-12.00) | (2.00-12.00) | (2.00-12.00) |
| | | | [n = 18] | |
| Cₘₐₓ (ng/mL) | 520 | 359 | 447 | 207 |
| | (136.7%) | (102.3%) | (86.6%) | (118.1%) |
| | | | [n = 18] | |
| AUC₍₀₋₂₄₎ | 1970 | 1710 | 1790 | 1090 |
| (ng · h/mL) | (170.6%) | (151.5%) | (49.0%) | (95.8%) |
| | [n = 17] | [n = 17] | [n = 11] | [n = 12] |
| AUC₍₀₋ₗₐₛₜ₎ | 1640 | 1420 | 1500 | 859 |
| (ng · h/mL) | (188.4%) | (164.9%) | (111.7%) | (163.8%) |
| | | | [n = 18] | |
| AUC_{(0-inf)} | 1510 | 1290 | 1860 | 1170 |
| (ng · h/mL) | (226.5%) | (160.1%) | (33.5%) | (83.1%) |
| | [n = 8] | [n = 8] | [n = 6] | [n = 7] |
| T_{1/2} (h) | 1.434 | 1.541 | 3.865 | 3.919 |
| | (58.4%) | (38.4%) | (84.7%) | (94.1%) |
| | [n = 8] | [n = 8] | [n = 6] | [n = 7] |
| MPR Cₘₐₓ | N/A | 0.506 | N/A | 0.299 |
| | | (46.4%) | | (41.0%) |
| | | | | [n = 18] |
| MPR AUC₍₀₋₂₄₎ | N/A | 0.641 | N/A | 0.380 |
| | | (39.5%) | | (33.0%) |
| | | [n = 16] | | [n = 11] |
| MPR AUC₍₀₋ₗₐₛₜ₎ | N/A | 0.634 | N/A | 0.376 |
| | | (41.0%) | | (42.4%) |
| | | | | [n = 18] |
| MPR AUC_{(0-inf)} | N/A | 0.698 | N/A | 0.303 |
| | | (54.8%) | | (24.5%) |
| | | [n = 6] | | [n = 5] |

AUC(0-24): area under the curve from time 0 to 24 house post-dose; AUC(0-inf): area under the curve from time 0 extrapolated to infinity; AUC(0-last): area under the curve from time 0 to the time of last measurable concentration Cmax: maximum observed concentration; MPR AUC(0-24): metabolite to parent ratio based on AUC(0-24); MPR AUC(0-inf): metabolite to parent ratio based on AUC(0-inf): MPR AUC(0-last): metabolite to parent ratio based on AUC(0-last); MPR Cmax: metabolite to parent ratio based on Cmax; n: number of participants with an observation; N: number of participants in the dataset; N/A: not applicable; QD: once daily; T_{1/2}: terminal elimination half-life; Tₘₐₓ: time of maximum observed concentration.
Regimen A (Part 1): raltegravir 400 mg single dose (Days 1 and 7); and belumosudil 200 mg QD (Days 3 to 8).
^{a}Median (range).

For Part 1 (Regimen A), peak and overall exposure levels of raltegravir were comparable when raltegravir was dosed with belumosudil tablet compared to dosing alone. However, for peak and overall exposure levels of raltegravir glucuronide, a notable decrease was observed when raltegravir was dosed with belumosudil compared to dosing alone. Belumosudil coadministration did not change the median time of maximum observed concentration (Tmax) of either raltegravir or raltegravir glucuronide. The geometric mean terminal elimination half-life (T½) values increased following belumosudil coadministration, from approximately 1.4 hours (raltegravir) or 1.5 hours (raltegravir glucuronide) on Day 1 to approximately 3.9 hours (both analytes) on Day 7.

For Regimen B (Part 2), geometric mean (geometric CV %) values for plasma PK parameters for unconjugated dabigatran are presented in Table 9, below.

TABLE 9

| Treatment | Dabigatran | Dabigatran |
|---|---|---|
| (Day) | etexilate | etexilate + |
| Number of | alone | belumosudil |
| participants | (Day 1) | (Day 9) |
| Parameter | N = 19 | N = 19 |
| Tₘₐₓ^{a} (h) | 3.000 | 2.000 |
| | (2.00-4.00) | (1.50-8.00) |
| Cₘₐₓ (ng/mL) | 42.1 | 99.5 |
| | (70.9%) | (110.1%) |
| AUC₍₀₋ₗₐₛₜ₎ | 317 | 682 |
| (ng · h/mL) | (67.6%) | (86.5%) |
| AUC_{(0-inf)} | 371 | 801 |
| (ng · h/mL) | (52.2%) | (73.2%) |
| | [n = 17] | [n = 17] |
| T_{1/2} (h) | 8.128 | 8.293 |
| | (18.3%) | (24.8%) |
| | [n = 18] | [n = 18] |

AUC(0-inf): area under the curve from time 0 extrapolated to infinity; AUC(0-last): area under the curve from time 0 to the time of last measurable concentration; Cmax: maximum observed concentration; n: number of participants with an observation; N: number of participants in the dataset; QD: once daily; T_{1/2}: terminal elimination half-life; Tmax: time of maximum observed concentration.
Regimen B (Part 2): dabigatran etexilate 75 mg single dose (Days 1 and 9); and belumosudil 200 mg QD (Days 5 to 12).
^{a}Median (range).

For Part 2 (Regimen B), peak and overall exposure levels of unconjugated dabigatran (following administration of dabigatran etexilate) were increased by 2.36- and 2.06-fold, respectively, when dosed with belumosudil compared to dosing alone. Median Tmax and geometric mean T_{1/2} were similar following administration of dabigatran etexilate alone or with belumosudil.

For Regimen C (Part 3), geometric mean (geometric CV %) values for plasma PK parameters for rosuvastatin are presented in Table 10, below.

TABLE 10

| Treatment | | Rosuvastatin |
|---|---|---|
| (Day) | Rosuvastatin | calcium + |
| Number of | calcium alone | belumosudil |
| participants | (Day 1) | (Day 10) |
| Parameter | N = 14 | N = 14 |
| Tₘₐₓ^{a} (h) | 6.000 | 2.500 |
| | (2.00-10.00) | (1.50-4.00) |
| | [n = 11] | |
| Cₘₐₓ (ng/mL) | 2.96 | 9.49 |
| | (79.1%) | (49.5%) |
| | [n = 11] | |
| AUC₍₀₋ₗₐₛₜ₎ | 13.6 | 54.0 |
| (ng · h/mL) | (75.3%) | (43.9%) |
| | [n = 10] | |
| AUC_{(0-inf)} | 25.4, 39.3^{b} | 54.1 |
| (ng · h/mL) | [n = 2] | (30.1%) |
| | | [n = 13] |
| T_{1/2} (h) | 4.050 | 2.748 |
| | (46.4%) | (20.4%) |
| | [n = 5] | [n = 13] |

AUC(0-inf): area under the curve from time 0 extrapolated to infinity; AUC(0-last): area under the curve from time 0 to the time of last measurable concentration; Cmax: maximum observed concentration; n: number of participants with an observation; N: number of participants in the dataset; QD: once daily; T_{1/2}: terminal elimination half-life; Tmax: time of maximum observed concentration.
Regimen C (Part 3): rosuvastatin calcium 10 mg single dose (Days 1 and 10) and belumosudil 200 mg QD (Days 6 to 13).
^{a}Median (range).
^{b}Individual participant value(s).

For Part 3 (Regimen C), peak and overall exposure levels of rosuvastatin (following administration of rosuvastatin calcium) were increased by 3.59- and 4.62-fold, respectively, when dosed with belumosudil compared to dosing alone. Additionally, belumosudil coadministration resulted in an earlier median Tmax (decrease from approximately 6 hours postdose on Day 1 to 2.5 hours postdose on Day 10), and geometric mean T_{1/2} decreased from 4.1 to 2.7 hours, although this change should be interpreted with caution due to the low number of reliable estimates (n=5) on Day 1 compared with Day 10 (n=13).

For Regimen A (Part 1), geometric mean (geometric CV %) values for PK parameters for belumosudil, KD025m1 and KD025m2 following oral doses of 200 mg belumosudil, alone and coadministered with 400 mg raltegravir (a prototypical UGT1A1 substrate) are presented in Table 11, below.

TABLE 11

| | Treatment (Day) | | | | | |
|---|---|---|---|---|---|---|
| | Belumosudil alone | | | Raltegravir + belumosudil | | |
| | (Day 3) | | | (Day 8) | | |
| | Analyte Number of participants | | | | | |
| | Belumosudil | KD025m1 | KD025m2 | Belumosudil | KD025m1 | KD025m2 |
| Parameter | N = 19 | N = 19 | N = 19 | N = 19 | N = 19 | N = 19 |
| Tₘₐₓ^{a} (h) | 2.00 | 2.00 | 2.00 | 2.00 | 1.50 | 3.00 |
| | (1.00-4.00) | (1.00-3.00) | (1.00-4.00) | (1.50-4.00) | (1.00-4.00) | (1.50-4.00) |
| | | [n = 17] | | | | |
| Cₘₐₓ (ng/mL) | 2130 | 21.4 | 278 | 2760 | 18.6 | 368 |
| | (36.6%) | (45.9%) | (100.2%) | (14.7%) | (41.7%) | (65.8%) |
| | | [n = 17] | | | | |
| AUC₍₀₋ₜₐᵤ₎ | 9330 | 96.6 | 961 | 13 000 | 90.7 | 1260 |
| (ng · h/mL) | (38.3%) | (12.0%) | (73.2%) | (21.9%) | (18.7%) | (64.4%) |
| | | [n = 13] | [n = 17] | | [n = 3] | |
| AUC₍₀₋ₗₐₛₜ₎ | 9200 | 47.6 | 879 | 13 700 | 36.9 | 1210 |
| (ng · h/mL) | (39.0%) | (41.6%) | (75.9%) | (22.6%) | (50.6%) | (69.4%) |
| | [n = 18] | [n = 13] | [n = 18] | | [n = 13] | |
| T_{1/2} (h) | 5.841 | 1.621 | 3.695 | 8.303 | 2.335 | 4.746 |
| | (27.0%) | (27.1%) | (48.3%) | (30.2%) | (25.0%) | (61.7%) |
| | [n = 16] | [n = 3] | [n = 14] | [n = 15] | [n = 3] | [n = 15] |

AUC(0-last): area under the curve from time 0 to the time of last measurable concentration; AUC(0-tau): area under the curve for the defined interval between doses (tau); Cmax: maximum observed concentration; n: number of participants with an observation; N: number of participants in the dataset; QD: once daily; T_{1/2}: terminal elimination half-life; Tmax: time of maximum observed concentration.
Regimen A (Part 1): raltegravir 400 mg single dose (Days 1 and 7) and belumosudil 200 mg QD (Days 3 to 8).
^{a}Median (range).

In Part 1, following single and repeated administration of belumosudil, median Tmax of belumosudil, KD025m1 and KD025m2 was attained at approximately 1.5 to 3 hours postdose, and exposures (Cmax and AUC) of belumosudil and KD025m2 increased by between 1.30- and 1.49-fold following multiple dosing.

Geometric mean T_{1/2} for belumosudil increased following repeated administration of belumosudil compared to a single dose, from approximately 6 hours on Day 3 to approximately 8 hours on Day 8; geometric mean T_{1/2} for KD025m1 was approximately 1.6 to 2 hours on Days 3 and 8, and geometric mean T_{1/2} for KD025m2 increased slightly from approximately 4 hours on Day 3 to approximately 5 hours on Day 8.

For Regimen B (Part 2), geometric mean (geometric CV %) values for PK parameters for belumosudil, KD025m1 and KD025m2 following oral doses of 200 mg belumosudil, alone and coadministered with 75 mg dabigatran etexilate (a prototypical P-gp substrate) are presented in Table 12, below.

TABLE 12

| | Treatment (Day) | | | | | |
|---|---|---|---|---|---|---|
| | Belumosudil alone | | | Dabigatran etexilate + belumosudil | | |
| | (Day 5) | | | (Day 12) | | |
| | Analyte Number of participants | | | | | |
| | Belumosudil | KD025m1 | KD025m2 | Belumosudil | KD025m1 | KD025m2 |
| Parameter | N = 19 | N = 19 | N = 19 | N = 19 | N = 19 | N = 19 |
| Tₘₐₓ^{a} (h) | 2.000 | 1.750 | 2.000 | 2.000 | 1.500 | 2.000 |
| | (1.50-6.00) | (1.50-4.00) | (1.50-6.00) | (1.00-6.00) | (0.50-30.00) | (1.50-30.00) |
| | | [n = 14] | [n = 18] | | [n = 15] | |
| Cₘₐₓ (ng/mL) | 1710 | 19.5 | 184 | 2180 | 18.9 | 236 |
| | (113.5%) | (44.6%) | (122.1%) | (142.1%) | (49.0%) | (117.8%) |
| | | [n = 14] | [n = 18] | | [n = 15] | |
| AUC₍₀₋ₜₐᵤ₎ | 8490 | 80.6, | 746 | 11 000 | 281b | 991 |
| (ng · h/mL) | (92.6%) | 184^{b} | (63.3%) | (98.5%) | [n = 1] | (63.0%) |
| | | [n = 2] | [n = 16] | | | [n = 17] |
| AUC₍₀₋ₗₐₛₜ₎ | 8430 | 35.3 | 692 | 12 000 | 38.4 | 921 |
| (ng · h/mL) | (96.0%) | (86.1%) | (66.4%) | (93.8%) | (94.9%) | (68.4%) |
| | | [n = 8] | [n = 16] | | [n = 7] | [n = 17] |
| T_{1/2} (h) | 6.199 | 1.878^{b} | 2.858 | 10.371 | N/C | 4.450 |
| | (31.4%) | [n = 1] | (54.9%) | (34.6%) | | (63.3%) |
| | [n = 11] | | [n = 14] | [n = 13] | | [n = 11] |

AUC(0-last): area under the curve from time 0 to the time of last measurable concentration; AUC(0-tau): area under the curve for the defined interval between doses (tau); Cmax: maximum observed concentration; n: number of participants with an observation; N: number of participants in the dataset; N/C: not calculated; QD: once daily; T_{1/2}: terminal elimination half-life; Tmax: time of maximum observed concentration.
Regimen B (Part 2): dabigatran etexilate 75 mg single dose (Days 1 and 9) and belumosudil 200 mg QD (Days 5 to 12).
^{a}Median (range).
^{b}Individual participant value(s).

In Part 2, following single and repeated administration of belumosudil, median Tmax of belumosudil, KD025m1 and KD025m2 was attained at approximately 1.5 to 2 hours postdose, and exposures (Cmax and AUC) of belumosudil and KD025m2 increased by between 1.27- and 1.42-fold following multiple dosing.

Where sufficient estimates were available, geometric mean T_{1/2} for belumosudil increased following repeated administration of belumosudil compared to a single dose, from approximately 6 hours on Day 5 to approximately 10 hours on Day 12; geometric mean T_{1/2} for KD025m2 increased slightly from approximately 3 hours on Day 5 to approximately 4 hours on Day 12.

For Regimen C (Part 3), geometric mean (geometric CV %) values for PK parameters for belumosudil, KD025m1 and KD025m2 following oral doses of 200 mg belumosudil, alone and coadministered with 10 mg rosuvastatin calcium (a prototypical BCRP/OATP1B1 substrate) are presented in Table 13, below.

TABLE 13

| | Treatment (Day) | | | | | |
|---|---|---|---|---|---|---|
| | Belumosudil alone | | | Dabigatran calcium + belumosudil | | |
| | (Day 6) | | | (Day 13) | | |
| | Analyte Number of participants | | | | | |
| | Belumosudil | KD025m1 | KD025m2 | Belumosudil | KD025m1 | KD025m2 |
| Parameter | N = 14 | N = 14 | N = 14 | N = 14 | N = 14 | N = 14 |
| Tₘₐₓ^{a} (h) | 2.000 | 1.500 | 2.000 | 2.000 | 1.500 | 2.000 |
| | (1.50-4.00) | (1.00-3.00) | (2.00-4.00) | (1.00-3.00) | (1.00-3.00) | (1.50-3.00) |
| | | [n = 12] | [n = 12] | [n = 13] | [n = 12] | [n = 12] |
| Cₘₐₓ (ng/mL) | 2080 | 17.1 | 215 | 2510 | 16.8 | 265 |
| | (20.7%) | (35.4%) | (69.3%) | (29.0%) | (43.3%) | (78.6%) |
| | | [n = 12] | [n = 12] | [n = 13] | [n = 12] | [n = 12] |
| AUC₍₀₋ₜₐᵤ₎ | 9710 | 96.9, | 598 | 12 900 | 116 | 937 |
| (ng · h/mL) | (27.8%) | 120^{b} | (72.4%) | (26.6%) | (46.3%) | (76.6%) |
| | | [n = 2] | [n = 12] | [n = 13] | [n = 3] | [n = 12] |
| AUC₍₀₋ₗₐₛₜ₎ | 9710 | 32.0 | 557 | 13 600 | 41.2 | 872 |
| (ng · h/mL) | (27.8%) | (61.4%) | (75.4%) | (27.4%) | (67.6%) | (83.0%) |
| | | [n = 7] | [n = 12] | [n = 13] | [n = 5] | [n = 12] |
| T_{1/2} (h) | 4.901 | 1.63, | 1.973 | 9.102 | 1.495^{b} | 6.337 |
| | (25.1%) | 3.18^{b} | (42.0%) | (26.7%) | [n = 1] | (93.3%) |
| | [n = 8] | [n = 2] | [n = 8] | [n = 12] | | [n = 6] |

AUC(0-last): area under the curve from time 0 to the time of last measurable concentration; AUC(0-tau): area under the curve for the defined interval between doses (tau); Cmax: maximum observed concentration; n: number of participants with an observation; N: number of participants in the dataset; QD: once daily; T_{1/2}: terminal elimination half-life; Tmax: time of maximum observed concentration.
Regimen C (Part 3): rosuvastatin calcium 10 mg single dose (Days 1 and 10) and belumosudil 200 mg QD (Days 6 to 13).
^{a}Median (range).
^{b}Individual participant value(s).

In Part 3, following single and repeated administration of belumosudil, median Tmax of belumosudil, KD025m1 and KD025m2 was attained at approximately 1.5 to 2 hours postdose, and exposures (Cmax and AUC) of belumosudil and KD025m2 increased by between 1.21- and 1.57-fold following multiple dosing.

Where sufficient estimates were available, geometric mean T_{1/2} for belumosudil increased following repeated administration of belumosudil compared to a single dose, from approximately 5 to 9 hours; and geometric mean T_{1/2} for KD025m2 increased from approximately 2 to 6 hours.

### Safety Results.

Overall, the perpetrator IMP, belumosudil, was well tolerated when coadministered with the victim IMPs raltegravir, dabigatran etexilate, and rosuvastatin calcium. No serious adverse events or treatment emergent adverse events (TEAEs) leading to participant withdrawal or death were reported, and the overall number of TEAEs and number of participants reporting TEAEs was low. Only 2 (3.8%) participants reported 1 TEAE each during the study, both of which were Grade 1 (mild) in severity and resolved without medication or intervention. Only 1 event (headache) was judged by the Investigator to be related to the perpetrator IMP (belumosudil); no TEAEs were judged to be related to the victim IMPs. There were no clinically significant findings in the clinical laboratory evaluations, vital signs measurements, ECGs or physical examination assessments for any regimen, and no trends were observed in these data. No new potential safety signals were observed after review of the data generated in this study.

Although the present invention has been described in some detail by way of illustration and example for purposes of clarity and understanding, the descriptions and examples should not be construed as limiting the scope of the invention. The disclosures of all patent and scientific literature cited herein are expressly incorporated herein in their entirety by reference.

1. A method of treating chronic graft-versus-host-disease (cGVHD) in a subject in need thereof, the method comprising co-administering at least one substrate of organic anion transporting polypeptide 1B1 (OATP1B1), breast cancer resistance protein (BCRP), or P-glycoprotein (P-gp) and 2-{3-[4-(1H-indazol-5-ylamino)-2-quinazolinyl]phenoxy}-N-(propan-2-yl) acetamide or a pharmaceutically acceptable salt thereof (Compound), wherein the dose of the at least one substrate of OATP1B1, BCRP, or P-gp is reduced as compared to the clinically-recommended dose when the substrate is administered without the Compound.

2. The method according to claim 1, wherein the Compound comprises the mesylate salt of 2-{3-[4-(1H-indazol-5-ylamino)-2-quinazolinyl]phenoxy}-N-(propan-2-yl) acetamide.

3. The method according to claim 1 or claim 2, wherein the dose of the Compound is about 200 mg.

4. The method according to any one of claims 1 to 3, wherein a substrate of OATP1B1 is co-administered to the subject.

5. The method according to claim 4, wherein the substrate of OATP1B1 is at least one of estradiol-17β-glucuronide, pitavastatin, pravastatin, rosuvastatin, atorvastatin, lovastatin, or simvastatin.

6. The method according to claim 5, wherein the substrate of OATP1B1 is rosuvastatin.

7. The method according to any one of claims 1 to 3, wherein a substrate of BCRP is co-administered to the subject.

8. The method according to claim 7, wherein the substrate of BCRP is rosuvastatin.

9. The method according to claim 8, wherein the co-administered dose of rosuvastatin does not exceed 5 mg daily.

10. The method according to any one of claims 1-3, wherein a substrate of P-gp is co-administered to the subject.

11. The method according to claim 10, wherein the substrate of P-gp is dabigatran etexilate.

12. The method according to claim 11, wherein the co-administered dose of dabigatran etexilate is less than about 300 mg.

13. The method according to claim 1, wherein the dose of the Compound is about 200 mg, and wherein the dose of the at least one substrate of OATP1B1, BRCP, or P-gp is reduced by about half as compared to the clinically-recommended dose when the substrate is administered without the Compound.

14. The method according to claim 13, wherein the at least one substrate of OATP1B1, BCRP, or P-gp is rosuvastatin.

15. The method according to claim 13 or claim 14, wherein the Compound comprises belumosudil mesylate salt.

16. The method according to any one of claims 1 to 15, wherein the subject has chronic graft-versus-host disease and has failed at least two prior lines of systemic therapy for the chronic graft-versus-host disease.

17. A method for treating a subject with cGVHD comprising the steps of:
(a) determining whether the subject is in need of co-administration of at least one substrate of OATP1B1, BCRP, or P-gp;
(b) determining the clinically-recommended dose of the at least one substrate of OATP1B1, BCRP, or P-gp for the subject when the substrate is administered without co-administration of 2-{3-[4-(1H-indazol-5-ylamino)-2-quinazolinyl]phenoxy}-N-(propan-2-yl) acetamide or a pharmaceutically acceptable salt thereof (Compound);
(c) determining a lower dose of the at least one substrate of OATP1B1, BCRP, or P-gp to be administered to the subject to mediate the increase in exposure of the substrate due to the administration of the Compound; and
(d) co-administering the at least one substrate of OATP1B1, BCRP, or P-gp and the Compound.

18. The method according to claim 17, wherein the Compound comprises belumosudil mesylate salt.

19. The method according to claim 17, wherein the at least one substrate of OATP1B1, BCRP, or P-gp is rosuvastatin.
